(19) United States
(12) Patent Application Publication
(10) Pub. No.: US 2002/0155114 A1
(43) Pub. Date: Oct. 24, 2002

(54) THERAPEUTIC MONOCLONAL ANTIBODIES THAT NEUTRALIZE BOTULINUM NEUROTOXINS

(76) Inventors: JAMES D. MARKS, KENSINGTON, CA (US); PETER AMERSDORFER, BOSTON, MA (US)

Correspondence Address: LAW OFFICES OF JONATHAN ALAN QUINE, P O BOX 458, ALAMEDA, CA, 94501

(21) Appl. No.: 09/144,886

(22) Filed: Aug. 31, 1998

(51) Int. Cl. A61K 39/40

(52) U.S. Cl. 424/150.1

(57) ABSTRACT

This invention provides antibodies that specifically bind to and neutralize botulinum neurotoxin type A (BoNT/A) and the epitopes bound by those antibodies. The antibodies and derivatives thereof and/or other antibodies that specifically bind to the neutralizing epitopes provided herein can be used to neutralize botulinum neurotoxin and are therefore also useful in the treatment of botulism.

### STATEMENT AS TO RIGHTS TO INVENTIONS MADE UNDER FEDERALLY SPONSORED RESEARCH AND DEVELOPMENT

This work was partially supported by the U.S. Army Medical Research and Development Command under award no. DAMD17-74-C-4034. The Government of the United States of America may have certain rights in this invention.

### CROSS-REFERENCE TO RELATED APPLICATIONS

[Not Applicable]

### FIELD OF THE INVENTION

This invention relates antibodies that neutralize botulinum neurotoxin type A (BoNT/A) and their use in the treatment of botulism.

### BACKGROUND OF THE INVENTION

Botulism is a life-threatening, flaccid paralysis caused by a neurotoxin produced by the anaerobic bacterium Clostridium botulinum. The disease typically results from ingestion of pre-formed toxin present in contaminated food (Dowell (1984) Rev. Infect. Dis. 6(Suppl. 1): S202-S207), from toxin produced in vivo from infected wounds (Weber (1993) Clin. Infect. Dis., 16: 635-639, in the intestines of infants (Arnon (1992) in Textbook of pediatric infectious diseases, R. D. Feigen and J. D. Cherry (ed.), 3rd ed., Saunders, Philadelphia, Pa.), or occasionally in adults.

In severe cases, patients require prolonged hospitalization in an intensive-care unit and mechanical ventilation. Specific therapy consists of administration of botulism antitoxin trivalent (equine) (Tacket et al. Am. J. Med., 76: 794-798); however, this product has a high incidence of side effects, including serum sickness and anaphylaxis (Black, et al. (1980) Am. J. Med., 69: 567-570). To avoid these side effects, human BIG has been produced from immunized volunteers and its efficacy is being determined in a prospective randomized trial in infants with botulism (Arnon (1993) pages 477-482 in Botulinum and tetanus neurotoxins: neurotransmission and biomedical aspects, B. R. DasGupta (ed.), Plenum, New York, N.Y.). While theoretically nontoxic, human BIG also has limitations, largely related to production issues. These include potential transmission of blood-borne infectious diseases, variability in potency and specificity between lots, and the need to immunize humans. The latter issue has taken on increased importance with the use of BoNTs for the treatment of a range of neuromuscular diseases (Jankovic et al. (1994) Therapy with botulinum toxin. Marcel Dekker, New York, N.Y.; Moore (1995) Handbook of botulinum toxin treatment, Blackwell Science, Oxford, United Kingdom). Immunization of volunteers for production of BIG would deprive them of subsequent botulinum therapy.

As an alternative to immune globulin, neutralizing monoclonal antibodies with defined potency and specificity could be produced in unlimited quantities. To date, however, no efficacious neutralizing antibotulinum monoclonal antibodies have been produced (Middlebrook, et al. (1995) Curr. Top. Microbiol. Immunol. 195:89-122). Potential explanations for this failure include the following: (i) a neutralizing epitope(s) is less immunogenic than other epitopes; (ii) too few unique monoclonal antibodies have been studied; (iii) a toxoid immunogen (formaldehyde-inactivated crude toxin) which poorly mimics the conformation of the neutralizing epitope(s) has been used; and (iv) multiple epitopes must be blocked in order to achieve efficient neutralization (Lang, et al. (1993) J. Immunol. 151: 466-473).

### SUMMARY OF THE INVENTION

This invention provides novel antibodies that specifically bind to and neutralize botulinum neurotoxin type A (BoNT/A). In addition, the epitopes bound by these antibodies are provided. The antibodies and epitopes identified herein are suitable for the creation of fully human, or humanized (chimeric) whole (polyclonal or monoclonal) antibodies and/or antibody fragments. In addition the antibodies and/or variants thereof are useful in neutralizing botulinum neurotoxin type A and can be used to mitigate or eliminate symptoms of botulism.

Thus, in one embodiment, this invention provides an isolated antibody that specifically binds to an epitope specifically bound by an antibody expressed by a clone selected from the group consisting of clone S25, clone C25, clone C39, clone 1C6, and clone 1F3. The antibody binds to and neutralizes botulinum neurotoxin type A (BoNT/A). The antibody can be of virtually any mammalian animal type (e.g. mouse, human, goat, rabbit) or chimeric (e.g. humanized), but is most preferably mouse, human, or humanized.

In one embodiment, the antibody comprises at least one (more preferably at least two and most preferably at least three) of the variable heavy (V_{H}) complementarity determining regions (CDRs) listed in Table 4 or conservative substitutions thereof. In another embodiment, the antibody comprises at least one (more preferably at least two and most preferably at least three) of the variable light (V_{L}) complementarity determining regions (CDRs) listed in Table 4 or conservative substitutions thereof. In still another embodiment, the antibody comprises at least one (more preferably at least two and most preferably at least three) of the variable heavy (V_{H}) complementarity determining regions (CDRs) listed in Table 4 or conservative substitutions thereof and at least one (more preferably at least two and most preferably at least three) of the variable light (V_{L}) complementarity determining regions (CDRs) listed in Table 4 or conservative substitutions thereof. Particularly preferred antibodies are antibodies expressed by a clone listed in Table 4 (or human or humanized variants thereof). Particularly preferred antibodies are a single chain Fv (scFv), while other preferred antibodies include, but are not limited to a Fab, a a (Fab′)₂, and a (scFv′)₂. The antibodies can include fusion proteins comprising of two scFv fragments. Particularly preferred antibodies comprise a framework (e.g., a V_{H} or V_{L} framework 1, framework 2, framework 3, framework 4 or combinations thereof (e.g., at least two, at least three, or four V_{L} or V_{H} frameworks)).region listed in Table 4. Other preferred embodiments include an an antibody comprising a variable heavy (V_{H}) complementarity determining region (CDR) listed in Table 4 and wherein said antibody specifically binds to and neutralizes a botulinum neurotoxin type A. Preferred antibodies include one or more of the V_{H} and/or V_{L} CDR and/or framework regions as described herein.

This invention also provides for pharmaceutical compositions comprising one or more of the botulinum neurotoxin type A (BoNT/A)-neutralizing antibodies described herein in a pharamcological excipient.

This invention also provides methods of neutralizing a botulinum neurotoxin type A (BoNT/A). The methods involve contacting the botulinum neurotoxin type A with one or more of the BoNT/A-neutralizing antibodies described herein. Preferred antibodies have a specificity and affinity such that they specifically binds to binds to and neutralizes the botulinum neurotoxin type A. The methods can further involve contacting the BoNT/A with a second BoNT/A-neutralizing antibody.

This invention also provides BoNT/A-neutralizing epitopes. Preferred epitopes are BoNT/A Hc epitopes specifically bound by an antibody expressed by clone S25, C25, C39, 1C6, or 1F3. Particularly preferred polypeptides are not a full-length BoNT/A and more particularly preferred polypeptides are not a full-length BoNT/A H_{c} fragment. Thus, most preferred epitopes are a BoNT/A H_{C} subsequence or fragment with preferred subsequences having a length of at least 4, preferably at least 6, more preferably at least 8 and most preferably at least 10, 12, 14, or even 15 amino acids.

This invention also provides methods of making a botulinum neurotoxin type A antibody (anti-BoNT/A) that neutralizes BoNT/A. The methods involve contacting a plurality of antibodies with an epitope specifically bound by an antibody expressed one or more of clones S25, C25, C39, 1C6, or 1F3. Particularly preferred epitopes are polypeptides that are not a full-length BoNT/A and more particularly preferred polypeptides are not a full-length BoNT/A H_{c} fragment. Thus, most preferred epitopes are a BoNT/A H_{C} subsequence or fragment with preferred subsequences having a length of at least 4, preferably at least 6, more preferably at least 8 and most preferably at least 10, 12, 14, or even 15 amino acids. The plurality of antibodies can include, but is not limited to antibodies displayed on a surface protein of a phage, and/or antibodies in serum from a mammal, and/or antibodies expressed by hybridomas.

Definitions

The following abbreviations are used herein: AMP, ampicillin; BIG, botulinum immune globulin; BoNT, botulinum neurotoxin; BoNT/A, BoNT type A; CDR, complementarity determining region; ELISA, enzyme-linked immunosorbent assay; GLU, glucose; HBS, HEPES-buffered saline (10 mM HEPES, 150 mM NaCl [pH 7.4]); H_{c}, c-terminal domain of BoNT heavy chain (binding domain); H_{N}, N-terminal domain of BoNT heavy chain (translocation domain); IgG, immunoglobutin G; IMAC, immobilized-metal affinity chromatography; IPTG, isopropyl-β-D-thiogalactopyranoside; KAN, kanamycin; K_{d}, equilibrium constant; k_{off}, dissociation rate constant; kₒₙ, association rate constant; MPBS, skim milk powder in PBS; NTA, nitrilotriacetic acid; PBS, phosphate-buffered saline (25 mM NaH₂PO₄, 125 mM NaCl [pH 7.0]; RU, resonance units; scFv, single-chain Fv antibody fragments; TPBS, 0.05% (vol/vol) Tween 20 in PBS; TMPBS, 0.05% (vol/vol) Tween 20 in MPBS; TU, transducing units; V_{H}, immunoglobulin heavy-chain variable region; V_{K}, immunoglobulin kappa light-chain variable region; V_{L} immunoglobulin light-chain variable region; wt, wild type.

The terms “polypeptide”, “peptide”, or “protein” are used interchangeably herein to designate a linear series of amino acid residues connected one to the other by peptide bonds between the alpha-amino and carboxy groups of adjacent residues. The amino acid residues are preferably in the natural “L” isomeric form. However, residues in the “D” isomeric form can be substituted for any L-amino acid residue, as long as the desired functional property is retained by the polypeptide. In addition, the amino acids, in addition to the 20 “standard” amino acids, include modified and unusual amino acids, which include, but are not limited to those listed in 37 CFR (1.822(b)(4). Furthermore, it should be noted that a dash at the beginning or end of an amino acid residue sequence indicates either a peptide bond to a further sequence of one or more amino acid residues or a covalent bond to a carboxyl or hydroxyl end group.

The term “BoNT/A polypeptide” refers to either a full-length BoNT/A or a fragment thereof (e.g. the Hc fragment). BoNT/A is a neurotoxin produced by Clostridium botulinum of the type A serotype. The H_{C} fragment is a 43 kDa C-terminal fragment (residues 860-1296) of BoNT/A (LaPenotiere et al. (1995) Toxicon, 33: 1383-1386).

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs”. Thus, the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface”. This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

As used herein, the terms “immunological binding” and “immunological binding properties” refer to the non-covalent interactions of the type which occur between an immunoglobulin molecule and an antigen for which the immunoglobulin is specific. The strength or affinity of immunological binding interactions can be expressed in terms of the dissociation constant (K_{d}) of the interaction, wherein a smaller Kd represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. Ann. Rev. Biochem., 59: 439-473 (1 990).

The term “BoNT/A-neutralizing antibody”, as used herein refers to an antibody that specifically binds to a BoNT/A polypeptide, more preferably to a BoNT/A H_{C} polypeptide and that by so-binding reduces the toxicity of the BoNT/A polypeptide. Reduced toxicity can be measured as an increase in the time that paralysis developed and/or as a lethal dosage (e.g. LD₅₀) as described herein. Antibodies derived from BoNT/A-neutralizing antibodies include the antibodies whose sequence is expressly provided herein.

Antibodies derived from BoNT/A-neutralizing antibodies preferably have a binding affinity of about 1.6×10⁻⁸ or better and are preferably derived by screening (for affinity to BoNT/A) a phage display library in which a known BoNT/A-neutralizing variable heavy (V_{H}) chain is expressed in combination with a multiplicity of variable light (V_{L}) chains or conversely a known BoNT/A-neutralizing variable light chain is expressed in combination with a multiplicity of variable heavy (V_{H}) chains. BoNT/A-neutralizing antibodies also include those antibodies produced by the introduction of mutations into the variable heavy or variable light complementarity determining regions (CDR1, CDR2 or CDR3) as described herein. Finally BoNT/A-neutralizing antibodies include those antibodies produced by any combination of these modification methods as applied to the BoNT/A-neutralizing antibodies described herein and their derivatives.

A neutralizing epitope refers to the epitope specifically bound by a neutralizing antibody.

A single chain Fv (“scFv” or “scFv”) polypeptide is a covalently linked V_{H}::V_{L} heterodimer which may be expressed from a nucleic acid including V_{H}- and V_{L}-encoding sequences either joined directly or joined by a peptide-encoding linker. Huston, et al. (1988) Proc. Nat. Acad. Sci. USA, 85: 5879-5883. A number of structures for converting the naturally aggregated—but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

In one class of embodiments, recombinant design methods can be used to develop suitable chemical structures (linkers) for converting two naturally associated—but chemically separate—heavy and light polypeptide chains from an antibody variable region into a scFv molecule which will fold into a three-dimensional structure that is substantially similar to native antibody structure.

Design criteria include determination of the appropriate length to span the distance between the C-terminal of one chain and the N-terminal of the other, wherein the linker is generally formed from small hydrophilic amino acid residues that do not tend to coil or form secondary structures. Such methods have been described in the art. See, e.g., U.S. Pat. Nos. 5,091,513 and 5,132,405 to Huston et al.; and U.S. Pat. No. 4,946,778 to Ladner et al.

In this regard, the first general step of linker design involves identification of plausible sites to be linked. Appropriate linkage sites on each of the V_{H} and V_{L} polypeptide domains include those which will result in the minimum loss of residues from the polypeptide domains, and which will necessitate a linker comprising a minimum number of residues consistent with the need for molecule stability. A pair of sites defines a “gap” to be linked. Linkers connecting the C-terminus of one domain to the N-terminus of the next generally comprise hydrophilic amino acids which assume an unstructured configuration in physiological solutions and preferably are free of residues having large side groups which might interfere with proper folding of the V_{H} and V_{L} chains. Thus, suitable linkers under the invention generally comprise polypeptide chains of alternating sets of glycine and serine residues, and may include glutamic acid and lysine residues inserted to enhance solubility. One particular linker under the invention has the amino acid sequence [(Gly)₄Ser]₃. Another particularly preferred linker has the amino acid sequence comprising 2 or 3 repeats of [(Ser)₄Gly] such as [(Ser)₄Gly]₃ Nucleotide sequences encoding such linker moieties can be readily provided using various oligonucleotide synthesis techniques known in the art. See, e.g., Sambrook, supra.

The phrase “specifically binds to a protein” or “specifically immunoreactive with”, when referring to an antibody refers to a binding reaction which is determinative of the presence of the protein in the presence of a heterogeneous population of proteins and other biologics. Thus, under designated immunoassay conditions, the specified antibodies bind to a particular protein and do not bind in a significant amount to other proteins present in the sample. Specific binding to a protein under such conditions may require an antibody that is selected for its specificity for a particular protein. For example, BoNT/A-neutralizing antibodies can be raised to the BoNT/A protein that specifically bind to BoNT/A and not to other proteins present in a tissue sample. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988) Antibodies, A Laboratory Manual, Cold Spring Harbor Publications, New York, for a description of immunoassay formats and conditions that can be used to determine specific immunoreactivity.

The term “conservative substitution” is used in reference to proteins or peptides to reflect amino acid substitutions that do not substantially alter the activity (specificity or binding affinity) of the molecule. Typically conservative amino acid substitutions involve substitution one amino acid for another amino acid with similar chemical properties (e.g. charge or hydrophobicity). The following six groups each contain amino acids that are typical conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

### BRIEF DESCRIPTION OF THE DRAWINGS

FIG. 1 Illustrates the strategy for in vitro antibody production using phage libraries. mRNA is prepared from splenocytes, first-strand cDNA is prepared, and antibody V_{H} and V_{L} genes are amplified by PCR. V_{H} and V_{L} genes are spliced together randomly using PCR to create a repertoire of scFv genes. The scFv gene repertoire is cloned into a phagemid vector in frame with a gene (gIII) encoding a phagemid minor coat protein (pIII). Each phage in the resulting phage antibody library expresses and scFv-pIII fusion protein on its surface and contains the gene encoding the scFv inside. Phage antibodies binding a specific antigen can be separated from nonbinding phage antibodies by affinity chromatography on immobilized antigen. A single round of selection increases the number of antigen-binding phage antibodies by a factor ranging from 20 to 10,000 depending on the affinity of the antibody. Eluted phage antibodies are used to infect E. coli, which then produce more phage antibodies for the next round of selection. Repeated rounds of selection make it possible to isolate antigen-binding phage antibodies that were originally present at frequencies of less than one in a billion.

FIG. 2 panel A and panel B show sensor grams illustrating the technique used to epitope map scFv binding to BoNT/A H_{C}. Epitope mapping was performed by using surface plasmon resonance in a BIAcore, with scFv studied in pairs. Each scFv was injected into the BIAcore and allowed to bind to BoNT/A H_{C} coupled to the sensor chip surface until saturation was achieved. The amount (in RU) bound for each scFv alone was compared to the amount bound when the two scFv were mixed and injected together. Point a shows the baseline, followed by the beginning of injection. Points b₁ and b₂ show the initial association phase. Points c₁ and c₂ show the beginning of dissociation. The differences in RU between points a and c equal the amount of scFv bound to BoNT/A H_{C}. Panel A shows two scFv recognizing different epitopes (C25 and C9). The amount bound of the two scFv injected together (C9/C25, point c₂) is the sum of the two scFv injected alone (c₁). Panel B shows two scFv recognizing the same epitope (C39 and C25). The amount bound for the two scFv injected together (C25/C39; point c) is the same as that for the two scFv injected alone (c). The large differences in RU between points b₁ and c₁, b₂ and c₂, and b₁ and c are due to differences in refractive index between scFv and running buffer.

FIG. 3 shows the evaluation of scFv neutralization of BoNT/A in a mouse hemidiaphragm model. The twitch tension developed after electrical stimulation of a mouse hemidiaphragm was measured below (−30 to 0 min) and after the addition of 20 pM BoNT/a (control), 2 pM BoNT/A plus 20 nM scFv S25, C25, 1BoNT/A-neutralizing, or 1F3 (representing epitopes 1 to 4 respectively), or a combination of S25 and C25 at a final concentration of 20 nM each. Results are expressed as the fraction of steady-state twitch tension (at 0 min) versus time. scFv 1C6 and 1F3 do not alter the time to 50% twitch reduction, whereas scFv C25 and S25 significantly prolong it. The combination of S25 and C25 significantly prolonged the time to neuroparalysis compared to C25 or S25 alone.

### DETAILED DESCRIPTION

This invention provides novel antibodies that specifically bind to and neutralize botulinum neurotoxin type A, a neurotoxin produced by the anaerobic bacterium Clostridium botulinum. Botulinum neurotoxin poisoning (botulism) arises in a number of contexts including, but not limited to food poisoning (food borne botulism), infected wounds (wound botulism), and “infant botulism” from ingestion of spores and production of toxin in the intestine of infants. Botulism is a paralytic disease that typically begins with cranial nerve involvement and progresses caudally to involve the extremities. In acute cases, botulism can prove fatal.

The antibodies provided by this invention bind to and neutralize botulinum neurotoxin type A (BoNT/A). Neutralization, in this context, refers to a measurable decrease in the toxicity of BoNT/A. Such a decrease in toxicity can be measured in vitro by a number of methods well known to those of skill in the art. One such assay involves measuring the time to a given percentage (e.g. 50%) twitch tension reduction in a hemidiaphragm preparation. Toxicity can be determined in vivo, e.g. as an LD₅₀ in a test animal (e.g. mouse) botulinum neurotoxin type A in the presence of one or more putative neutralizing antibodies. The neutralizing antibody can be combined with the botulinum neurotoxin prior to administration, or the animal can be administered the antibody prior to, simultaneous with, or after administration of the neurotoxin.

As the antibodies of this invention act to neutralize botulinum neurotoxin type A, they are useful in the treatment of pathologies associated with botulinum neurotoxin poisoning. The treatments essentially comprise administering to the poisoned organism (e.g. human or non-human mammal) a quantity of BoNT/A neutralizing antibody sufficient to neutralize (e.g. mitigate or eliminate) symptoms of BoNT/A poisoning.

Such treatments are most desired and efficacious in acute cases (e.g. where vital capacity is less than 30-40 percent of predicted and/or paralysis is progressing rapidly and/or hypoxemia with absolute or relative hypercarbia is present. Treatment with the neutralizing can be provided as a adjunct to other therapies (e.g. antibiotic treatment).

The antibodies provided by this invention can also be used for the rapid detection/diagnosis of botulism (type A toxin) and thereby supplement and/or replace previous laboratory diagnostics.

In another embodiment this invention provides the epitopes specifically bound by botulinum neurotoxin type A neutralizing antibodies. These epitopes can be used to isolate, and/or identify and/or screen for other antibodies BoNT/A neutralizing antibodies as described herein.

I. Preparation of BoNT/A Neutralizing Antibodies

A) Recombinant Expression of BoNT/A-neutralizing Antibodies

Using the information provided herein, the botulinum neurotoxin type A (BoNT/A)-neutralizing antibodies of this invention are prepared using standard techniques well known to those of skill in the art.

For example, the polypeptide sequences provided herein (see, e.g., Table 4) may be used to determine appropriate nucleic acid sequences encoding the BoNT/A-neutralizing antibodies and the nucleic acids sequences then used to express one or more BoNT/A-neutralizing antibodies. The nucleic acid sequence may be optimized to reflect particular codon “preferences” for various expression systems according to standard methods well known to those of skill in the art.

Using the sequence information provided, the nucleic acids may be synthesized according to a number of standard methods known to those of skill in the art. Oligonucleotide synthesis, is preferably carried out on commercially available solid phase oligonucleotide synthesis machines (Needham-VanDevanter et al. (1984) Nucleic Acids Res. 12:6159-6168) or manually synthesized using the solid phase phosphoramidite triester method described by Beaucage et. al. (Beaucage et. al. (1981) Tetrahedron Letts. 22(20): 1859-1862).

Once a nucleic acid encoding a BoNT/A-neutralizing antibody is synthesized it may be amplified and/or cloned according to standard methods. Molecular cloning techniques to achieve these ends are known in the art. A wide variety of cloning and in vitro amplification methods suitable for the construction of recombinant nucleic acids are known to persons of skill. Examples of these techniques and instructions sufficient to direct persons of skill through many cloning exercises are found in Berger and Kimmel, Guide to Molecular Cloning Techniques, Methods in Enzymology volume 152 Academic Press, Inc., San Diego, Calif. (Berger); Sambrook et al. (1989) Molecular Cloning—A Laboratory Manual (2nd ed.) Vol. 1-3, Cold Spring Harbor Laboratory, Cold Spring Harbor Press, NY, (Sambrook); and Current Protocols in Molecular Biology, F. M. Ausubel et al., eds., Current Protocols, a joint venture between Greene Publishing Associates, Inc. and John Wiley & Sons, Inc., (1994 Supplement) (Ausubel). Methods of producing recombinant immunoglobulins are also known in the art. See, Cabilly, U.S. Pat. No. 4,816,567; and Queen et al. (1989) Proc. Nat'l Acad. Sci. USA 86: 10029-10033.

Examples of techniques sufficient to direct persons of skill through in vitro amplification methods, including the polymerase chain reaction (PCR) the ligase chain reaction (LCR), Qβ-replicase amplification and other RNA polymerase mediated techniques are found in Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomell et al. (1989) J. Clin. Chem 35, 1826; Landegren et al., (1988) Science 241, 1077-1080; Van Brunt (1990) Biotechnology 8, 291-294; Wu and Wallace, (1989) Gene 4, 560; and Barringer et al. (1990) Gene 89, 117. Improved methods of cloning in vitro amplified nucleic acids are described in Wallace et al., U.S. Pat. No. 5,426,039.

Once the nucleic acid for a BoNT/A-neutralizing antibody is isolated and cloned, one may express the gene in a variety of recombinantly engineered cells known to those of skill in the art. Examples of such cells include bacteria, yeast, filamentous fungi, insect (especially employing baculoviral vectors), and mammalian cells. It is expected that those of skill in the art are knowledgeable in the numerous expression systems available for expression of BoNT/A-neutralizing antibodies.

In brief summary, the expression of natural or synthetic nucleic acids encoding BoNT/A-neutralizing antibodies will typically be achieved by operably linking a nucleic acid encoding the antibody to a promoter (which is either constitutive or inducible), and incorporating the construct into an expression vector. The vectors can be suitable for replication and integration in prokaryotes, eukaryotes, or both. Typical cloning vectors contain transcription and translation terminators, initiation sequences, and promoters useful for regulation of the expression of the nucleic acid encoding the BoNT/A-neutralizing antibody. The vectors optionally comprise generic expression cassettes containing at least one independent terminator sequence, sequences permitting replication of the cassette in both eukaryotes and prokaryotes, i.e., shuttle vectors, and selection markers for both prokaryotic and eukaryotic systems. See Sambrook.

To obtain high levels of expression of a cloned nucleic acid it is common to construct expression plasmids which typically contain a strong promoter to direct transcription, a ribosome binding site for translational initiation, and a transcription/translation terminator. Examples of regulatory regions suitable for this purpose in E. coli are the promoter and operator region of the E. coli tryptophan biosynthetic pathway as described by Yanofsky, (1984) J. Bacteriol., 158:1018-1024 and the leftward promoter of phage lambda (P_{L}) as described by Herskowitz and Hagen (1980) Ann. Rev. Genet., 14:399-445. The inclusion of selection markers in DNA vectors transformed in E. coli is also useful. Examples of such markers include genes specifying resistance to ampicillin, tetracycline, or chloramphenicol. See Sambrook for details concerning selection markers, e.g., for use in E. coli.

Expression systems for expressing BoNT/A-neutralizing antibodies are available using E. coli, Bacillus sp. (Palva, et al. (1983) Gene 22:229-235; Mosbach et al., Nature, 302: 543-545 and Salmonella. E. coli systems are preferred.

The BoNT/A-neutralizing antibodies produced by prokaryotic cells may require exposure to chaotropic agents for proper folding. During purification from, e.g., E. coli, the expressed protein is optionally denatured and then renatured. This is accomplished, e.g., by solubilizing the bacterially produced antibodies in a chaotropic agent such as guanidine HCl. The antibody is then renatured, either by slow dialysis or by gel filtration. See, U.S. Pat. No. 4,511,503.

Methods of transfecting and expressing genes in mammalian cells are known in the art. Transducing cells with nucleic acids can involve, for example, incubating viral vectors containing BoNT/A-neutralizing nucleic acids with cells within the host range of the vector. See, e.g., Goeddel (1990) Methods in Enzymology, vol. 185, Academic Press, Inc., San Diego, Calif. or Krieger (1990) Gene Transfer and Expression—A Laboratory Manual, Stockton Press, New York, N.Y. and the references cited therein.

The culture of cells used in the present invention, including cell lines and cultured cells from tissue or blood samples is well known in the art (see, e.g., Freshney (1994) Culture of Animal Cells, a Manual of Basic Technique, third edition, Wiley-Liss, N. Y. and the references cited therein).

Techniques for using and manipulating antibodies are found in Coligan (1991) Current Protocols in Immunology Wiley/Greene, NY; Harlow and Lane (1989) Antibodies: A Laboratory Manual Cold Spring Harbor Press, NY; Stites et al. (eds.) Basic and Clinical Immunology (4th ed.) Lange Medical Publications, Los Altos, Calif., and references cited therein; Goding (1986) Monoclonal Antibodies: Principles and Practice (2d ed.) Academic Press, New York, NY; and Kohler and Milstein (1975) Nature 256: 495-497. BoNT/A-neutralizing antibodies that are specific for botulinum neurotoxin type A have a K_{D} of 1×10⁻⁸ M or better, with preferred embodiments having a K_{D} of 1 nM or better and most preferred embodiments having a K_{D} of 0.1 nM or better.

In one preferred embodiment the BoNT/A-neutralizing antibody gene (e.g. BoNT/A-neutralizing.5 scFv gene) is subcloned into the expression vector pUC119mycHis (Tomlinson et al. (1996) J. Mol. Biol., 256: 813-817) or pSYN3, resulting in the addition of a hexahistidine tag at the C-terminal end of the scFv to facilitate purification. Detailed protocols for the cloning and purification of BoNT/A-neutralizing antibodies are provided in Example 1.

B) Preparation of Whole Polyclonal or Monoclonal Antibodies

BoNT/A-neutralizing antibodies of this invention include individual, allelic, strain, or species variants, and fragments thereof, both in their naturally occurring (full-length) forms and in recombinant forms. Preferred antibodies are selected to bind one or more epitopes bound by antibodies expressed by clones S25, C25, C39, 1C6, and 1F3 disclosed herein. The antibodies can be raised in their native configurations or in non-native configurations. Anti-idiotypic antibodies can also be generated. Many methods of making antibodies that specifically bind to a particular epitope are known to persons of skill. The following discussion is presented as a general overview of the techniques available; however, one of skill will recognize that many variations upon the following methods are known.

1) Polyclonal antibody production.

Methods of producing polyclonal antibodies are known to those of skill in the art. In brief, an immunogen (e.g., BoNT/A, BoNT/A H_{c}, or BoNT/A subsequences including, but not limited to subsequences comprising epitopes specifically bound by antibodies expressed by clones S25, C25, C39, 1C6, and 1F3 disclosed herein), preferably a purified polypeptide, a polypeptide coupled to an appropriate carrier (e.g., GST, keyhole limpet hemanocyanin, etc.), or a polypeptide incorporated into an immunization vector such as a recombinant vaccinia virus (see, U.S. Pat. No. 4,722,848) is mixed with an adjuvant and animals are immunized with the mixture. The animal's immune response to the immunogen preparation is monitored by taking test bleeds and determining the titer of reactivity to the polypeptide of interest. When appropriately high titers of antibody to the immunogen are obtained, blood is collected from the animal and antisera are prepared. Further fractionation of the antisera to enrich for antibodies reactive to the BoNT/A polypeptide is performed where desired (see, e.g., Coligan (1991) Current Protocols in Immunology Wiley/Greene, NY; and Harlow and Lane (1989) Antibodies: A Laboratory Manual Cold Spring Harbor Press, NY).

Antibodies that specifically bind to the neutralizing epitopes described herein can be selected from polyclonal sera using the selection techniques described herein.

2) Monoclonal antibody production.

In some instances, it is desirable to prepare monoclonal antibodies from various mammalian hosts, such as mice, rodents, primates, humans, etc. Descriptions of techniques for preparing such monoclonal antibodies are found in, e.g., Stites et al. (eds.) Basic and Clinical Immunology (4th ed.) Lange Medical Publications, Los Altos, Calif., and references cited therein; Harlow and Lane, supra; Goding (1986) Monoclonal Antibodies: Principles and Practice (2d ed.) Academic Press, New York, N.Y.; and Kohler and Milstein (1975) Nature 256: 495-497.

Summarized briefly, monoclonal antibody production proceeds by injecting an animal with an immunogen (e.g., BoNT/A, BoNT/A H_{c}, or BoNT/A subsequences including, but not limited to subsequences comprising epitopes specifically bound by antibodies expressed by clones S25, C25, C39, 1C6, and 1F3 disclosed herein). The animal is then sacrificed and cells taken from its spleen, which are fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the BoNT/A antigen, and yield of the monoclonal antibodies produced by such cells is enhanced by various techniques, including injection into the peritoneal cavity of a vertebrate (preferably mammalian) host. The antibodies of the present invention are used with or without modification, and include chimeric antibodies such as humanized murine antibodies.

II. Modification of BoNT/A Neutralizing Antibodies

A) Phage Display can be used to Increase Antibody Affinity

To create higher affinity antibodies, mutant scFv gene repertories, based on the sequence of a binding scFv (e.g. Table 4), are created and expressed on the surface of phage. Display of antibody fragments on the surface of viruses which infect bacteria (bacteriophage or phage) makes it possible to produce human or other mammalian antibodies (e.g. scFvs) with a wide range of affinities and kinetic characteristics. To display antibody fragments on the surface of phage (phage display), an antibody fragment gene is inserted into the gene encoding a phage surface protein (e.g., pIII) and the antibody fragment-pIII fusion protein is expressed on the phage surface (McCafferty et al. (1990) Nature, 348: 552-554; Hoogenboom et al. (1991) Nucleic Acids Res., 19: 4133-4137).

Since the antibody fragments on the surface of the phage are functional, those phage bearing antigen binding antibody fragments can be separated from non-binding or lower affinity phage by antigen affinity chromatography (McCafferty et al. (1990) Nature, 348: 552-554). Mixtures of phage are allowed to bind to the affinity matrix, non-binding or lower affinity phage are removed by washing, and bound phage are eluted by treatment with acid or alkali. Depending on the affinity of the antibody fragment, enrichment factors of 20 fold-1,000,000 fold are obtained by single round of affinity selection.

By infecting bacteria with the eluted phage or modified variants of the eluted phage as described below, more phage can be grown and subjected to another round of selection. In this way, an enrichment of 1000 fold in one round becomes 1,000,000 fold in two rounds of selection (McCafferty et al. (1990) Nature, 348: 552-554). Thus, even when enrichments in each round are low, multiple rounds of affinity selection leads to the isolation of rare phage and the genetic material contained within which encodes the sequence of the binding antibody (Marks et al. (1991) J. Mol. Biol., 222: 581-597). The physical link between genotype and phenotype provided by phage display makes it possible to test every member of an antibody fragment library for binding to antigen, even with libraries as large as 100,000,000 clones. For example, after multiple rounds of selection on antigen, a binding scFv that occurred with a frequency of only 1/30,000,000 clones was recovered (Id.).

1) Chain shuffling.

One approach for creating mutant scFv gene repertoires involves replacing either the V_{H} or V_{L} gene from a binding scFv with a repertoire of V_{H} or V_{L} genes (chain shuffling) (Clackson et al. (1991) Nature, 352: 624-628). Such gene repertoires contain numerous variable genes derived from the same germline gene as the binding scFv, but with point mutations (Marks et al. (1992) Bio/Technology, 10: 779-783). Using light or heavy chain shuffling and phage display, the binding avidities of BoNT/A-neutralizing antibody fragment can be dramatically increased (see, e.g., Marks et al. (1992) Bio/Technology, 10: 779-785 in which the affinity of a human scFv antibody fragment which bound the hapten phenyloxazolone (phox) was increased from 300 nM to 15 nM (20 fold)).

Thus, to alter the affinity of BoNT/A-neutralizing antibody a mutant scFv gene repertoire is created containing the V_{H} gene of a known BoNT/A-neutralizing antibody (see Table 4) and a V_{L} gene repertoire (light chain shuffling). Alternatively, an scFv gene repertoire is created containing the V_{L} gene of a known BoNT/A-neutralizing antibody (see Table 4) and a V_{H} gene repertoire (heavy chain shuffling). The scFv gene repertoire is cloned into a phage display vector (e.g., pHEN-1, Hoogenboom et al. (1991) Nucleic Acids Res., 19: 4133-4137) and after transformation a library of transformants is obtained. Phage were prepared and concentrated and selections are performed as described in the examples.

The antigen concentration is decreased in each round of selection, reaching a concentration less than the desired K_{d} by the final rounds of selection. This results in the selection of phage on the basis of affinity (Hawkins et al. (1992) J. Mol. Biol. 226: 889-896).

2) Increasing the affinity of BoNT/A-neutralizing antibodies by site directed mutagenesis.

The majority of antigen contacting amino acid side chains are located in the complementarity determining regions (CDRs), three in the V_{H} (CDR1, CDR2, and CDR3) and three in the V_{L} (CDR1, CDR2, and CDR3) (Chothia et al. (1987) J. Mol. Biol., 196: 901-917; Chothia et al. (1986) Science, 233: 755-8; Nhan et al. (1991) J. Mol. Biol., 217: 133-151). These residues contribute the majority of binding energetics responsible for antibody affinity for antigen. In other molecules, mutating amino acids that contact ligand has been shown to be an effective means of increasing the affinity of one protein molecule for its binding partner (Lowman et al. (1993) J. Mol. Biol., 234: 564-578; Wells (1990) Biochemistry, 29: 8509-8516). Thus mutation (randomization) of the CDRs and screening against BoNT/A, BoNT/A H_{C} or the epiotpes thereof identified herein, may be used to generate BoNT/A-neutralizing antibodies having improved binding affinity.

In a preferred embodiment, each CDR is randomized in a separate library, using, for example, S25 as a template (K_{d}=7.3×10⁻⁸ M). To simplify affinity measurement, S25, or other lower affinity BoNT/A-neutralizing antibodies, are used as a template, rather than a higher affinity scFv. The CDR sequences of the highest affinity mutants from each CDR library are combined to obtain an additive increase in affinity. A similar approach has been used to increase the affinity of human growth hormone (hGH) for the growth hormone receptor over 1500 fold from 3.4×10⁻¹⁰ to 9.0×10⁻¹³ M (Lowman et al. (1993) J. Mol. Biol., 234: 564-578).

To increase the affinity of BoNT/A-neutralizing antibodies, amino acid residues located in one or more CDRs (e.g. 9 amino acid residues located in V_{L} CDR3) are partially randomized by synthesizing a ‘doped’ oligonucleotide in which the wild type nucleotide occurred with a frequency of, e.g. 49%. The oligonucleotide is used to amplify the remainder of the BoNT/A-neutralizing scFv gene(s) using PCR.

For example in one embodiment, to create a library in which V_{H} CDR3 is randomized an oligonucleotide is synthesized which anneals to the BoNT/A-neutralizing antibody V_{H} framework 3 and encodes V_{H} CDR3 and a portion of framework 4. At the four positions to be randomized, the sequence NNS can be used, where N is any of the 4 nucleotides, and S is “C” or “T”. The oligonucleotide is used to amplify the BoNT/A-neutralizing antibody V_{H} gene using PCR, creating a mutant BoNT/A-neutralizing antibody V_{H} gene repertoire. PCR is used to splice the V_{H} gene repertoire with the BoNT/A-neutralizing antibody light chain gene, and the resulting scFv gene repertoire cloned into a phage display vector (e.g., pHEN-1 or pCANTAB5E). Ligated vector DNA is used to transform electrocompetent E. coli to produce a phage antibody library.

To select higher affinity mutant scFv, each round of selection of the phage antibody libraries is conducted on decreasing amounts of BoNT/A, as described in the Examples. Typically, 96 clones from the third and fourth round of selection are screened for binding to the BoNT/A antigen by ELISA on 96 well plates. scFv from twenty to forty ELISA positive clones are expressed, e.g. in 10 ml cultures, the periplasm harvested, and the scFv k_{off} determined by BIAcore. Clones with the slowest k_{off} are sequenced, and each unique scFv subcloned into an appropriate vector (e.g., pUC119 mycHis). The scFv are expressed in culture, and purified as described herein. Affinities of purified scFv are determined by BIAcore.

3) Creation of BoNT/A-neutralizing (scFv′)2 homodimers.

To create BoNT/A-neutralizing (scFv′)₂ antibodies, two BoNT/A-neutralizing scFvs are joined, either through a linker (e.g., a carbon linker, a peptide, etc.) or through a disulfide bond between, for example, two cysteins. Thus, for example, to create disulfide linked BoNT/A-neutralizing scFv, a cysteine residue can be introduced by site directed mutagenesis between the myc tag and hexahistidine tag at the carboxy-terminus of the BoNT/A-neutralizing scFv. Introduction of the correct sequence is verified by DNA sequencing. In a preferred embodiment, the construct is in pUC119, so that the pe1B leader directs expressed scFv to the periplasm and cloning sites (Ncol and Notl) exist to introduce BoNT/A-neutralizing mutant scFv. Expressed scFv has the myc tag at the C-terminus, followed by two glycines, a cysteine, and then 6 histidines to facilitate purification by IMAC. After disulfide bond formation between the two cysteine residues, the two scFv are separated from each other by 26 amino acids (two 11 amino acid myc tags and 4 glycines). An scFv was expressed from this construct, purified by IMAC may predominantly comprise monomeric scFv. To produce (scFv′)₂ dimers, the cysteine is reduced by incubation with 1 MM beta-mercaptoethanol, and half of the scFv blocked by the addition of DTNB. Blocked and unblocked scFvs are incubated together to form (scFv′)₂ and the resulting material can optionally be analyzed by gel filtration. The affinity of the BoNT/A-neutralizing scFv′ monomer and (scFv′)₂ dimer can optionally be determined by BIAcore as described herein.

In a particularly preferred embodiment, the (scFv′)₂ dimer is created by joining the scFv fragments through a linker, more preferably through a peptide linker. This can be accomplished by a wide variety of means well known to those of skill in the art. For example, one preferred approach is described by Holliger et al. (1993) Proc. Natl. Acad. Sci. USA, 90: 6444-6448 (see also WO 94/13804).

Typically, linkers are introduced by PCR cloning. For example, synthetic oligonucleotides encoding the 5 amino acid linker (G₄S) can be used to PCR amplify the BoNT/A-neutralizing antibody V_{H} and V_{L} genes which are then spliced together to create the BoNT/A-neutralizing diabody gene. The gene is then cloned into an appropriate vector, expressed, and purified according to standard methods well known to those of skill in the art.

4) Preparation of BoNT/A-neutralizing (scFv)₂, Fab, and (Fab′)₂ molecules.

BoNT/A-neutralizing antibodies such as BoNT/A-neutralizing scFv, or variant(s) with higher affinity, are suitable templates for creating size and valency variants. For example, a BoNT/A-neutralizing (scFv′)₂ is created from the parent scFv as described above. An scFv gene can be excised using appropriate restriction enzymes and cloned into another vector as described herein.

In one embodiment, expressed scFv has a myc tag at the C-terminus, followed by two glycines, a cysteine, and six histidines to facilitate purification. After disulfide bond formation between the two cystine residues, the two scFv should be separated from each other by 26 amino acids (e.g., two eleven amino acid myc tags and four glycines). scFv is expressed from this construct and purified.

To produce (scFv′)₂ dimers, the cysteine is reduced by incubation with 1 mM β-mercaptoethanol, and half of the scFv blocked by the addition of DTNB. Blocked and unblocked scFv are incubated together to form (scFv′)₂, which is purified. As higher affinity scFv are isolated, their genes are similarly used to construct (scFv′)₂.

BoNT/A-neutralizing Fab are expressed in E. coli using an expression vector similar to the one described by Better et. al. (1988) Science, 240: 1041-1043. To create a BoNT/A-neutralizing Fab, the V_{H} and V_{L} genes are amplified from the scFv using PCR. The V_{H} gene is cloned into an expression vector (e.g., a PUC119 based bacterial expression vector) that provides an IgG C_{H}1 domain downstream from, and in frame with, the V_{H} gene. The vector also contains the lac promoter, a pe1b leader sequence to direct expressed V_{H}-C_{H}1 domain into the periplasm, a gene 3 leader sequence to direct expressed light chain into the periplasm, and cloning sites for the light chain gene. Clones containing the correct V_{H} gene are identified, e.g., by PCR fingerprinting. The V_{L} gene is spliced to the C_{L} gene using PCR and cloned into the vector containing the V_{H} C_{H}1 gene.

B) Selection of Neutralizing Antibodies

In preferred embodiments, selection of BoNT/A-neutralizing antibodies (whether produced by phage display, immunization methods, hybridoma technology, etc.) involves screening the resulting antibodies for specific binding to an appropriate antigen. In the instant case, the preferred antigen is BoNT/A H_{C}, a C-terminal domain of BoNT heavy chain (binding domain). In particularly preferred embodiments the neutralizing antibodies are selected for specific binding of an epitope recognized by an antibody expressed by one or more of clones S25, C25, C39, 1C6, and 1F3.

Selection can by any of a number of methods well known to those of skill in the art. In a preferred embodiment, selection is by immunochromatography (e.g., using immunotubes, Maxisorp, Nunc) against BoNT/A or BoNT/A H_{C}. In another preferred embodiment, selection is against BoNT/A HC in surface plasmon resonance system (e.g. BIAcore, Pharmacia) either alone or in combination with an antibody that binds to an epitope specifically bound by an antibody expressed by one or more of clones S25, C25, C39, 1C6, and 1F3.

Analysis of binding can be simplified by including an amber codon between the antibody fragment gene and gene III. This makes it possible to easily switch between displayed and soluble antibody fragments simply by changing the host bacterial strain. When phage are grown in a supE suppresser strain of E. coli, the amber stop codon between the antibody gene and gene III is read as glutamine and the antibody fragment is displayed on the surface of the phage. When eluted phage are used to infect a non-suppressor strain, the amber codon is read as a stop codon and soluble antibody is secreted from the bacteria into the periplasm and culture media (Hoogenboom et al. (1991) Nucleic Acids Res., 19: 4133-4137). Binding of soluble scFv to antigen can be detected, e.g., by ELISA using a murine IgG monoclonal antibody (e.g., 9E1O) which recognizes a C-terminal myc peptide tag on the scFv (Evan et al. (1985) Mol. Cell Biol., 5: 3610-3616; Munro et al. (1986) Cell, 46: 291-300), e.g., followed by incubation with polyclonal anti-mouse Fc conjugated to a detectable label (e.g., horseradish peroxidase).

As indicated above, purification of the BoNT/A-neutralizing antibody can be facilitated by cloning of the scFv gene into an expression vector (e.g., expression vector pUC119mycHIS) that results in the addition of the myc peptide tag followed by a hexa-histidine tag at the C-terminal end of the scFv. The vector also preferably encodes the pectate lyase leader sequence that directs expression of the scFv into the bacterial periplasm where the leader sequence is cleaved. This makes it possible to harvest native properly folded scFv directly from the bacterial periplasm. The BoNT/A-neutralizing antibody is then expressed and purified from the bacterial supernatant using immobilized metal affinity chromatography.

C) Measurement of BoNT/A-neutralizing Antibody Affinity for BoNT/A

As explained above, selection for increased avidity involves measuring the affinity of a BoNT/A-neutralizing antibody (or a modified BoNT/A-neutralizing antibody) for BoNT/A (or a BoNT/A fragment (e.g., H_{c}), or an epitope on BoNT/A, etc.). Methods of making such measurements are described in detail in the examples provided herein. Briefly, for example, the K_{d} of a BoNT/A-neutralizing antibody and the kinetics of binding to BoNT/A are determined in a BIAcore, a biosensor based on surface plasmon resonance. For this technique, antigen is coupled to a derivatized sensor chip capable of detecting changes in mass. When antibody is passed over the sensor chip, antibody binds to the antigen resulting in an increase in mass that is quantifiable. Measurement of the rate of association as a function of antibody concentration can be used to calculate the association rate constant (kₒₙ). After the association phase, buffer is passed over the chip and the rate of dissociation of antibody (k_{off}) determined. Kₒₙ is typically measured in the range 1.0×10² to 5.0×10⁶ and k_{off} in the range 1.0×10⁻¹ to 1.0×10⁻⁶. The equilibrium constant K_{d} is then calculated as k_{off}/kₒₙ and thus is typically measured in the range 10⁻⁵ to 10⁻¹². Affinities measured in this manner correlate well with affinities measured in solution by fluorescence quench titration.

Phage display and selection generally results in the selection of higher affinity mutant scFvs (Marks et al. (1992) Bio/Technology, 10: 779-783; Hawkins et al. (1992) J. Mol. Biol. 226: 889-896; Riechmann et al. (1993) Biochemistry, 32: 8848-8855; Clackson et al. (1991) Nature, 352: 624-628), but probably does not result in the separation of mutants with less than a 6 fold difference in affinity (Riechmann et al. (1993) Biochemistry, 32: 8848-8855). Thus a rapid method is needed to estimate the relative affinities of mutant scFvs isolated after selection. Since increased affinity results primarily from a reduction in the k_{off}, measurement of k_{off} should identify higher affinity scFv. k_{off} can be measured in the BIAcore on unpurified scFv in bacterial periplasm, since expression levels are high enough to give an adequate binding signal and k_{off} is independent of concentration. The value of k_{off} for periplasmic and purified scFv is typically in close agreement.

III. Human or Humanized (Chimeric) Antibody Production

As indicated above, the BoNT/A-neutralizing antibodies of this invention can be administered to an organism (e.g., a human patient) for therapeutic purposes (e.g., the treatment of botulism). Antibodies administered to an organism other than the species in which they are raised can be immunogenic. Thus, for example, murine antibodies repeatedly administered to a human often induce an immunologic response against the antibody (e.g., the human anti-mouse antibody (HAMA) response). While this is typically not a problem for the use of non-human antibodies of this invention as they are typically not utilized repeatedly, the immunogenic properties of the antibody are reduced by altering portions, or all, of the antibody into characteristically human sequences thereby producing chimeric or human antibodies, respectively.

A) Humanized (Chimeric) Antibodies

Humanized (chimeric) antibodies are immunoglobulin molecules comprising a human and non-human portion. More specifically, the antigen combining region (or variable region) of a humanized chimeric antibody is derived from a non-human source (e.g., murine) and the constant region of the chimeric antibody (which confers biological effector function to the immunoglobulin) is derived from a human source. The humanized chimeric antibody should have the antigen binding specificity of the non-human antibody molecule and the effector function conferred by the human antibody molecule. A large number of methods of generating chimeric antibodies are well known to those of skill in the art (see, e.g., U.S. Pat. Nos: 5,502,167, 5,500,362, 5,491,088, 5,482,856, 5,472,693, 5,354,847, 5,292,867, 5,231,026, 5,204,244, 5,202,238, 5,169,939, 5,081,235, 5,075,431, and 4,975,369).

In general, the procedures used to produce chimeric antibodies consist of the following steps (the order of some steps may be interchanged): (a) identifying and cloning the correct gene segment encoding the antigen binding portion of the antibody molecule; this gene segment (known as the VDJ, variable, diversity and joining regions for heavy chains or VJ, variable, joining regions for light chains (or simply as the V or variable region) may be in either the cDNA or genomic form; (b) cloning the gene segments encoding the constant region or desired part thereof; (c) ligating the variable region to the constant region so that the complete chimeric antibody is encoded in a transcribable and translatable form; (d) ligating this construct into a vector containing a selectable marker and gene control regions such as promoters, enhancers and poly(A) addition signals; (e) amplifying this construct in a host cell (e.g., bacteria); (f) introducing the DNA into eukaryotic cells (transfection) most often mammalian lymphocytes; and culturing the host cell under conditions suitable for expression of the chimeric antibody.

Antibodies of several distinct antigen binding specificities have been manipulated by these protocols to produce chimeric proteins (e.g., anti-TNP: Boulianne et al. (1984) Nature, 312: 643; and anti-tumor antigens: Sahagan et al. (1986) J. Immunol., 137: 1066). Likewise several different effector functions have been achieved by linking new sequences to those encoding the antigen binding region. Some of these include enzymes (Neuberger et al. (1984) Nature 312: 604), immunoglobulin constant regions from another species and constant regions of another immunoglobulin chain (Sharon et al. (1984) Nature 309: 364; Tan et al., (1985) J. Immunol. 135: 3565-3567).

In one preferred embodiment, a recombinant DNA vector is used to transfect a cell line that produces a BoNT/A-neutralizing antibody. The novel recombinant DNA vector contains a “replacement gene” to replace all or a portion of the gene encoding the immunoglobulin constant region in the cell line (e.g., a replacement gene may encode all or a portion of a constant region of a human immunoglobulin, a specific immunoglobulin class, or an enzyme, a toxin, a biologically active peptide, a growth factor, inhibitor, or a linker peptide to facilitate conjugation to a drug, toxin, or other molecule, etc.), and a “target sequence” which allows for targeted homologous recombination with immunoglobulin sequences within the antibody producing cell.

In another embodiment, a recombinant DNA vector is used to transfect a cell line that produces an antibody having a desired effector function, (e.g., a constant region of a human immunoglobulin) in which case, the replacement gene contained in the recombinant vector may encode all or a portion of a region of an BoNT/A-neutralizing antibody and the target sequence contained in the recombinant vector allows for homologous recombination and targeted gene modification within the antibody producing cell. In either embodiment, when only a portion of the variable or constant region is replaced, the resulting chimeric antibody may define the same antigen and/or have the same effector function yet be altered or improved so that the chimeric antibody may demonstrate a greater antigen specificity, greater affinity binding constant, increased effector function, or increased secretion and production by the transfected antibody producing cell line, etc.

Regardless of the embodiment practiced, the processes of selection for integrated DNA (via a selectable marker), screening for chimeric antibody production, and cell cloning, can be used to obtain a clone of cells producing the chimeric antibody.

Thus, a piece of DNA which encodes a modification for a monoclonal antibody can be targeted directly to the site of the expressed immunoglobulin gene within a B-cell or hybridoma cell line. DNA constructs for any particular modification may be used to alter the protein product of any monoclonal cell line or hybridoma. Such a procedure circumvents the costly and time consuming task of cloning both heavy and light chain variable region genes from each B-cell clone expressing a useful antigen specificity. In addition to circumventing the process of cloning variable region genes, the level of expression of chimeric antibody should be higher when the gene is at its natural chromosomal location rather than at a random position. Detailed methods for preparation of chimeric (humanized) antibodies can be found in U.S. Pat. No. 5,482,856.

B) Human Antibodies

In another embodiment, this invention provides for fully human anti-BoNT/A-neutralizing antibodies. Human antibodies consist entirely of characteristically human polypeptide sequences. The human BoNT/A-neutralizing antibodies of this invention can be produced in using a wide variety of methods (see, e.g., Larrick et al., U.S. Pat. No. 5,001,065, for review).

In one preferred embodiment, fully human antibodies are produced using phage display methods as described herein. However, instead of utilizing a murine gene library, a human gene library is used. Methods of producing fully human gene libraries are well known to those of skill in the art (see, e.g., Vaughn et al. (1996) Nature Biotechnology, 14(3): 309-314, Marks et al. (1991) J. Mol. Biol., 222: 581-597, and PCT/US96/10287).

In another preferred embodiment, the human BoNT/A-neutralizing antibodies of the present invention are usually initially in trioma cells. Genes encoding the antibodies are then cloned and expressed in other cells, particularly, nonhuman mammalian cells.

The general approach for producing human antibodies by trioma technology has been described by Ostberg et al. (1983) Hybridoma 2: 361-367, Ostberg, U.S. Pat. No. 4,634,664, and Engelman et al., U.S. Pat. No. 4,634,666. The antibody-producing cell lines obtained by this method are called triomas because they are descended from three cells; two human and one mouse. Triomas have been found to produce antibody more stably than ordinary hybridomas made from human cells.

Preparation of trioma cells requires an initial fusion of a mouse myeloma cell line with unimmunized human peripheral B lymphocytes. This fusion generates a xenogenic hybrid cell containing both human and mouse chromosomes (see, Engelman, supra.). Xenogenic cells that have lost the capacity to secrete antibodies are selected. Preferably, a xenogenic cell is selected that is resistant to 8-azaguanine. Such cells are unable to propagate on hypoxanthine-aminopterin-thymidine (HAT) or azaserine-hypoxanthine (AH) media.

The capacity to secrete antibodies is conferred by a further fusion between the xenogenic cell and B-lymphocytes immunized against an BoNT/A polypeptide (e.g., BoNT/A, BoNT/A H_{c}, or BoNT/A subsequences including, but not limited to subsequences comprising epitopes specifically bound by antibodies expressed by clones S25, C25, C39, 1C6, and 1F3 disclosed herein). The B-lymphocytes are obtained from the spleen, blood or lymph nodes of human donor. If antibodies against a specific antigen or epitope are desired, it is preferable to use that antigen or epitope thereof as the immunogen rather than the entire polypeptide. Alternatively, B-lymphocytes are obtained from an unimmunized individual and stimulated with a BoNT/A polypeptide, or a epitope thereof, in vitro. In a further variation, B-lymphocytes are obtained from an infected, or otherwise immunized individual, and then hyperimmunized by exposure to a BoNT/A polypeptide for about seven to fourteen days, in vitro.

The immunized B-lymphocytes prepared by one of the above procedures are fused with a xenogenic hybrid cell by well known methods. For example, the cells are treated with 40-50% polyethylene glycol of MW 1000-4000, at about 37° C. for about 5-10 min. Cells are separated from the fusion mixture and propagated in media selective for the desired hybrids. When the xenogenic hybrid cell is resistant to 8-azaguanine, immortalized trioma cells are conveniently selected by successive passage of cells on HAT or AH medium. Other selective procedures are, of course, possible depending on the nature of the cells used in fusion. Clones secreting antibodies having the required binding specificity are identified by assaying the trioma culture medium for the ability to bind to the BoNT/A polypeptide or an epitope thereof. Triomas producing human antibodies having the desired specificity are subcloned by the limiting dilution technique and grown in vitro in culture medium, or are injected into selected host animals and grown in vivo.

The trioma cell lines obtained are then tested for the ability to bind a BoNT/A polypeptide or an epitope thereof. Antibodies are separated from the resulting culture medium or body fluids by conventional antibody-fractionation procedures, such as ammonium sulfate precipitation, DEAE cellulose chromatography and affinity chromatography.

Although triomas are genetically stable they do not produce antibodies at very high levels. Expression levels can be increased by cloning antibody genes from the trioma into one or more expression vectors, and transforming the vector into a cell line such as the cell lines typically used for expression of recombinant or humanized immunoglobulins. As well as increasing yield of antibody, this strategy offers the additional advantage that immunoglobulins are obtained from a cell line that does not have a human component, and does not therefore need to be subjected to the especially extensive viral screening required for human cell lines.

The genes encoding the heavy and light chains of immunoglobulins secreted by trioma cell lines are cloned according to methods, including but not limited to, the polymerase chain reaction (PCR), known in the art (see, e.g., Sambrook et al., Molecular Cloning: A Laboratory Manual, 2nd ed., Cold Spring Harbor, N.Y., 1989; Berger & Kimmel, Methods in Enzymology, Vol. 152: Guide to Molecular Cloning Techniques, Academic Press, Inc., San Diego, Calif., 1987; Co et al. (1992) J. Immunol., 148: 1149). For example, genes encoding heavy and light chains are cloned from a trioma's genomic DNA or cDNA produced by reverse transcription of the trioma's RNA. Cloning is accomplished by conventional techniques including the use of PCR primers that hybridize to the sequences flanking or overlapping the genes, or segments of genes, to be cloned.

Typically, recombinant constructs comprise DNA segments encoding a complete human immunoglobulin heavy chain and/or a complete human immunoglobulin light chain of an immunoglobulin expressed by a trioma cell line. Alternatively, DNA segments encoding only a portion of the primary antibody genes are produced, which portions possess binding and/or effector activities. Other recombinant constructs contain segments of trioma cell line immunoglobulin genes fused to segments of other immunoglobulin genes, particularly segments of other human constant region sequences (heavy and/or light chain). Human constant region sequences can be selected from various reference sources, including but not limited to those listed in Kabat et al. (1987) Sequences of Proteins of Immunological Interest, U.S. Department of Health and Human Services.

In addition to the DNA segments encoding BoNT/A-neutralizing immunoglobulins or fragments thereof, other substantially homologous modified immunoglobulins can be readily designed and manufactured utilizing various recombinant DNA techniques known to those skilled in the art such as site-directed mutagenesis (see Gillman & Smith (1979) Gene, 8: 81-97; Roberts et al. (1987) Nature 328: 731-734). Such modified segments will usually retain antigen binding capacity and/or effector function. Moreover, the modified segments are usually not so far changed from the original trioma genomic sequences to prevent hybridization to these sequences under stringent conditions. Because, like many genes, immunoglobulin genes contain separate functional regions, each having one or more distinct biological activities, the genes may be fused to functional regions from other genes to produce fusion proteins (e.g., immunotoxins) having novel properties or novel combinations of properties.

The genomic sequences can be cloned and expressed according to standard methods as described herein.

Other approaches to antibody production include in vitro immunization of human blood. In this approach, human blood lymphocytes capable of producing human antibodies are produced. Human peripheral blood is collected from the patient and is treated to recover mononuclear cells. The suppressor T-cells then are removed and remaining cells are suspended in a tissue culture medium to which is added the antigen and autologous serum and, preferably, a nonspecific lymphocyte activator. The cells then are incubated for a period of time so that they produce the specific antibody desired. The cells then can be fused to human myeloma cells to immortalize the cell line, thereby to permit continuous production of antibody (see U.S. Pat. No. 4,716,111).

In another approach, mouse-human hybridomas which produces human BoNT/A-neutralizing antibodies are prepared (see, e.g., U.S. Pat. No. 5,506,132). Other approaches include immunization of murines transformed to express human immunoglobulin genes, and phage display screening (Vaughan et al. supra.).

IV. Assaying for Cross-reactivity at a Neutralizing Epitope

In a preferred embodiment, the antibodies of this invention specifically bind to one or more epitopes recognized by antibodies expressed by clones S25, C25, C39, 1C6, or 1F3 (for convenience referred to herein as S25, C25, C39, 1C6, or 1F3 antibodies respectively). In other words, particularly preferred antibodies are cross-reactive with one of more of these antibodies. Means of assaying for cross-reactivity are well known to those of skill in the art (see, e.g., Dowbenko et al. (1988) J. Virol. 62: 4703-4711).

This can be ascertained by providing an isolated BoNT/A polypeptide (preferably BoNT/A Hc) attached to a solid support and assaying the ability of a test antibody to compete with S25, C25, C39, 1C6, or 1F3 antibodies for BoNT/A binding. Thus, immunoassays in a competitive binding format are preferably used for crossreactivity determinations. For example, in one embodiment, the BoNT/A H_{C} polypeptide is immobilized to a solid support. Antibodies to be tested (e.g. generated by selection from a phage-display library) added to the assay compete with S25, C25, C39, 1C6, or 1F3 antibodies binding to the immobilized BoNT/A polypeptide. The ability of test antibodies to compete with the binding of the S25, C25, C39, 1C6, or 1F3 antibodies to the immobilized protein are compared. The percent crossreactivity above proteins is then calculated, using standard calculations.

If the test antibody competes with one or more of the S25, C25, C39, 1C6, or 1F3 antibodies and has a binding affinity comparable to or greater than about 1×10⁻⁸ M with the same target then the test antibody will prove to be a BoNT/A neutralizing antibody.

In a particularly preferred embodiment, cross-reactivity is performed by using surface plasmon resonance in a BIAcore. In a BlAcore flow cell, the BoNT/A H_{c} is coupled to a sensor chip (e.g. CM5) as described in the examples. With a flow rate of 5 μl/min, a titration of 100 nM to 1 μM antibody is injected over the flow cell surface for about 5 minutes to determine an antibody concentration that results in near saturation of the surface. Epitope mapping or cross-reactivity is then evaluated using pairs of antibodies at concentrations resulting in near saturation and at least 100 RU of antibody bound. The amount of antibody bound is determined for each member of a pair, and then the two antibodies are mixed together to give a final concentration equal to the concentration used for measurements of the individual antibodies. Antibodies recognizing different epitopes show an essentially additive increase in the RU bound when injected together, while antibodies recognizing identical epitopes show only a minimal increase in RU (see the examples). In a particularly preferred embodiment, antibodies are said to be cross-reactive if, when “injected” together they show an essentially additive increase (preferably an increase by at least a factor of about 1.4, more preferably an increase by at least a factor of about 1.6, and most preferably an increase by at least a factor of about 1.8 or 2.

Cross-reactivity at the S25, C25, C39, 1C6, or 1F3 epitopes can ascertained by a number of other standard techniques (see, e.g., Geysen et al (1987) J. Immunol. Meth. 102, 259-274). This technique involves the synthesis of large numbers of overlapping BoNT/A H_{C} peptides. The synthesized peptides are then screened against one or more of the S25, C25, C39, 1C6, or 1F3 antibodies and the characteristic epitopes specifically bound by these antibodies can be identified by binding specificity and affinity. The epitopes thus identified can be conveniently used for competitive assays as described herein to identify cross-reacting antibodies.

The peptides for S25, C25, C39, 1C6, or 1F3 epitope mapping can be conveniently prepared using “Multipin” peptide synthesis techniques (see, e.g., Geysen et al (1987) Science, 235: 1184-1190). Using the known sequence of BoNT/A H_{C} (see, e.g., Atassi et al. (1996) J. Prot. Chem., 7: 691-700 and references cited therein), overlapping BoNT/A H_{C} polypeptide sequences can be synthesized individually in a sequential manner on plastic pins in an array of one or more 96-well microtest plate(s).

The procedure for epitope mapping using this multipin peptide system is described in U.S. Pat. No. 5,739,306. Briefly, the pins are first treated with a pre-coat buffer containing 2% bovine serum albumin and 0.1% Tween 20 in PBS for 1 hour at room temperature. Then the pins are then inserted into the individual wells of 96-well microtest plate containing antibody S25, C25, C39, 1C6, or 1F3 in the pre-coat buffer, e.g. at 2 mu g/ml. The incubation is preferably for about 1 hour at room temperature. The pins are washed in PBST (e.g., 3 rinses for every 10 minutes), and then incubated in the wells of a 96-well microtest plate containing 100 mu l of HRP-conjugated goat anti-mouse IgG (Fc) (Jackson ImmunoResearch Laboratories) at a 1:4,000 dilution for 1 hour at room temperature. After the pins are washed as before, the pins are put into wells containing peroxidase substrate solution of diammonium 2,2′-azino-bis[3-ethylbenzthiazoline-b-sulfonate] (ABTS) and H₂O₂ (Kirkegaard & Perry Laboratories Inc., Gaithersburg, Md.) for 30 minutes at room temperature for color reaction. The plate is read at 405 nm by a plate reader (e.g., BioTek ELISA plate reader) against a background absorption wavelength of 492 nm. Wells showing color development indicated reactivity of the BoNT/A H_{C} peptides in such wells with S25, C25, C39, 1C6, or 1F3 antibodies.

V. Assaying for Neutralizing Activity of Anti-BoNT/A Antibodies

Preferred antibodies of this invention act to neutralize (reduce or eliminate) the toxicity of botulinum neurotoxin type A. Neutralization can be evaluated in vivo or in vitro. In vivo neutralization measurements simply involve measuring changes in the lethality (e.g. LD₅₀ or other standard metric) due to a BoNT/A type A neurotoxin administration due to the presence of one or more antibodies being tested for neutralizing activity. The neurotoxin can be directly administered to the test organism (e.g. mouse) or the organism can harbor a botulism infection (e.g., be infected with Clostridium botulinum). The antibody can be administered before, during, or after the injection of BoNT/A neurotoxin or infection of the test animal. A decrease in the rate of progression, or mortality rate indicates that the antibody(s) have neutralizing activity.

A preferred in vitro assay for neutralizing activity uses a hemidiaphragm preparation (Deshpande et al. (1995) Toxicon, 33: 551-557). Briefly, left and right phrenic nerve hemidiaphragm preparations are suspended in physiological solution and maintained at a constant temperature (e.g. 36° C.). The phrenic nerves are stimulated supramaximally (e.g. at 0.05 Hz with square waves of 0.2 ms duration). Isometric twitch tension is measured with a force displacement transducer (e.g., GrassModel FT03) connected to a chart recorder.

Purified antibodies are incubated with purified BoNT/A for 30 min at room temperature and then added to the tissue bath, resulting in a final antibody concentration of about 2.0×10⁻⁸ M and a final BoNT/A concentration of about 2.0×10⁻¹¹ M. For each antibody studied, time to 50% twitch tension reduction is determined (e.g., three times for BoNT/A alone and three times for antibody plus BoNT/A). Differences between times to a given (arbitrary) percentage (e.g. 50%) twitch reduction are determined by standard statistical analyses (e.g. two-tailed t test) at standard levels of significance (e.g., a P value of <0.05 considered significant).

VI. Diagnostic Assays

As explained above, the BoNT/A-neutralizing antibodies may be used for the in vivo or in vitro detection of BoNT/A toxin and thus, are useful in the diagnosis (e.g. confirmatory diagnosis) of botulism. The detection and/or quantification of BoNT/A in a biological sample obtained from an organism is indicative of a Clostridium botulinum infection of that organism.

The BoNT/A antigen may be quantified in a biological sample derived from a patient such as a cell, or a tissue sample derived from a patient. As used herein, a biological sample is a sample of biological tissue or fluid that contains a BoNT/A concentration that may be correlated with and indicative of a Clostridium botulinum infection. Preferred biological samples include blood, urine, saliva, and tissue biopsies.

Although the sample is typically taken from a human patient, the assays can be used to detect BoNT/A antigen in cells from mammals in general, such as dogs, cats, sheep, cattle and pigs, and most particularly primates such as humans, chimpanzees, gorillas, macaques, and baboons, and rodents such as mice, rats, and guinea pigs.

Tissue or fluid samples are isolated from a patient according to standard methods well known to those of skill in the art, most typically by biopsy or venipuncture. The sample is optionally pretreated as necessary by dilution in an appropriate buffer solution or concentrated, if desired. Any of a number of standard aqueous buffer solutions, employing one of a variety of buffers, such as phosphate, Tris, or the like, at physiological pH can be used.

A) Immunological Binding Assays

The BoNT/A polypeptide is preferably detected in an immunoassay utilizing a BoNT/A-neutralizing antibody as a capture agent that specifically binds to the BoNT/A polypeptide.

As used herein, an immunoassay is an assay that utilizes an antibody (e.g. a BoNT/A-neutralizing antibody) to specifically bind an analyte (e.g., BoNT/A). The immunoassay is characterized by the use of specific antibody binding to a BoNT/A-neutralizing antibody as opposed to other physical or chemical properties to isolate, target, and quantify the BoNT/A analyte.

The BoNT/A marker may be detected and quantified using any of a number of well recognized immunological binding assays. (See for example, U.S. Pat. Nos. 4,366,241; 4,376,110; 4,517,288; and 4,837,168, which are hereby incorporated by reference.) For a review of the general immunoassays, see also Methods in Cell Biology Volume 37: Antibodies in Cell Biology, Asai, ed. Academic Press, Inc. New York (1993); Basic and Clinical Immunology 7th Edition, Stites & Terr, eds. (1991)).

The immunoassays of the present invention are performed in any of several configurations, e.g., those reviewed in Maggio (ed.) (1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B. V., Amsterdam; Harlow and Lane, supra; Chan (ed.) (1987) Immunoassay: A Practical Guide Academic Press, Orlando, Fla.; Price and Newman (eds.) (1991) Principles and Practice of Immunoassays Stockton Press, NY; and Ngo (ed.) (1988) Non isotopic Immunoassays Plenum Press, NY.

Immunoassays often utilize a labeling agent to specifically bind to and label the binding complex formed by the capture agent and the analyte (i.e., a BoNT/A-neutralizing antibody/ BoNT/A complex). The labeling agent may itself be one of the moieties comprising the antibody/analyte complex. Thus, the labeling agent may be a labeled BoNT/A or a labeled BoNT/A-neutralizing antibody. Alternatively, the labeling agent is optionally a third moiety, such as another antibody, that specifically binds to the BoNT/A-neutralizing antibody, the BoNT/A peptide, the anti-body/polypeptide complex, or to a modified capture group (e.g., biotin) which is covalently linked to BoNT/A or to the BoNT/A-neutralizing antibody.

In one embodiment, the labeling agent is an antibody that specifically binds to the BoNT/A-neutralizing antibody. Such agents are well known to those of skill in the art, and most typically comprise labeled antibodies that specifically bind antibodies of the particular animal species from which the BoNT/A-neutralizing antibody is derived (e.g., an anti-species antibody). Thus, for example, where the capture agent is a human derived BoNT/A-neutralizing antibody, the label agent may be a mouse anti-human IgG, i.e., an antibody specific to the constant region of the human antibody.

Other proteins capable of specifically binding immunoglobulin constant regions, such as streptococcal protein A or protein G are also used as the labeling agent. These proteins are normal constituents of the cell walls of streptococcal bacteria. They exhibit a strong non immunogenic reactivity with immunoglobulin constant regions from a variety of species. See, generally Kronval, et al., (1973) J. Immunol., 111:1401-1406, and Akerstrom, et al., (1985) J. Immunol., 135:2589-2542.

Throughout the assays, incubation and/or washing steps may be required after each combination of reagents. Incubation steps can vary from about 5 seconds to several hours, preferably from about 5 minutes to about 24 hours. However, the incubation time will depend upon the assay format, analyte, volume of solution, concentrations, and the like. Usually, the assays are carried out at ambient temperature, although they can be conducted over a range of temperatures, such as 5° C. to 45° C.

1) Non competitive assay formats.

Immunoassays for detecting BoNT/A are preferably either competitive or noncompetitive. Noncompetitive immunoassays are assays in which the amount of captured analyte (in this case, BoNT/A) is directly measured. In one preferred “sandwich” assay, for example, the capture agent (e.g., BoNT/A-neutralizing antibody) is bound directly or indirectly to a solid substrate where it is immobilized. These immobilized BoNT/A-neutralizing antibodies capture BoNT/A present in a test sample (e.g., a blood sample). The BoNT/A thus immobilized is then bound by a labeling agent, such as a BoNT/A-neutralizing antibody bearing a label. Alternatively, the second antibody may lack a label, but it may, in turn, be bound by a labeled third antibody specific to antibodies of the species from which the second antibody is derived. Free labeled antibody is washed away and the remaining bound labeled antibody is detected (e.g., using a gamma detector where the label is radioactive).

2) Competitive assay formats.

In competitive assays, the amount of analyte (e.g., BoNT/A) present in the sample is measured indirectly by measuring the amount of an added (exogenous) analyte displaced (or competed away) from a capture agent (e.g., BoNT/A-neutralizing antibody) by the analyte present in the sample. In one competitive assay, a known amount of BoNT/A is added to a test sample with an unquantified amount of BoNT/A, and the sample is contacted with a capture agent, e.g., a BoNT/A-neutralizing antibody that specifically binds BoNT/A. The amount of added BoNT/A that binds to the BoNT/A-neutralizing antibody is inversely proportional to the concentration of BoNT/A present in the test sample.

The BoNT/A-neutralizing antibody can be immobilized on a solid substrate. The amount of BoNT/A bound to the BoNT/A-neutralizing antibody is determined either by measuring the amount of BoNT/A present in an BoNT/A-BoNT/A-neutralizing antibody complex, or alternatively by measuring the amount of remaining uncomplexed BoNT/A.

B) Reduction of Non Specific Binding

One of skill will appreciate that it is often desirable to reduce non specific binding in immunoassays and during analyte purification. Where the assay involves BoNT/A, BoNT/A-neutralizing antibody, or other capture agent immobilized on a solid substrate, it is desirable to minimize the amount of non specific binding to the substrate. Means of reducing such non specific binding are well known to those of skill in the art. Typically, this involves coating the substrate with a proteinaceous composition. In particular, protein compositions such as bovine serum albumin (BSA), nonfat powdered milk, and gelatin are widely used.

C) Substrates

As mentioned above, depending upon the assay, various components, including the BoNT/A, BoNT/A-neutralizing or antibodies, are optionally bound to a solid surface. Many methods for immobilizing biomolecules to a variety of solid surfaces are known in the art. For instance, the solid surface may be a membrane (e.g., nitrocellulose), a microtiter dish (e.g., PVC, polypropylene, or polystyrene), a test tube (glass or plastic), a dipstick (e.g. glass, PVC, polypropylene, polystyrene, latex, and the like), a microcentrifuge tube, or a glass, silica, plastic, metallic or polymer bead. The desired component may be covalently bound, or noncovalently attached through nonspecific bonding.

A wide variety of organic and inorganic polymers, both natural and synthetic may be employed as the material for the solid surface. Illustrative polymers include polyethylene, polypropylene, poly(4-methylbutene), polystyrene, polymethacrylate, poly(ethylene terephthalate), rayon, nylon, poly(vinyl butyrate), polyvinylidene difluoride (PVDF), silicones, polyformaldehyde, cellulose, cellulose acetate, nitrocellulose, and the like. Other materials which may be employed, include paper, glasses, ceramics, metals, metalloids, semiconductive materials, cements or the like. In addition, substances that form gels, such as proteins (e.g., gelatins), lipopolysaccharides, silicates, agarose and polyacrylamides can be used. Polymers which form several aqueous phases, such as dextrans, polyalkylene glycols or surfactants, such as phospholipids, long chain (12-24 carbon atoms) alkyl ammonium salts and the like are also suitable. Where the solid surface is porous, various pore sizes may be employed depending upon the nature of the system.

In preparing the surface, a plurality of different materials may be employed, e.g., as laminates, to obtain various properties. For example, protein coatings, such as gelatin can be used to avoid non specific binding, simplify covalent conjugation, enhance signal detection or the like.

If covalent bonding between a compound and the surface is desired, the surface will usually be polyfunctional or be capable of being polyfunctionalized. Functional groups which may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature. See, for example, Immobilized Enzymes, Ichiro Chibata, Halsted Press, New York, 1978, and Cuatrecasas, (1970) J. Biol. Chem. 245 3059.

In addition to covalent bonding, various methods for noncovalently binding an assay component can be used. Noncovalent binding is typically nonspecific absorption of a compound to the surface. Typically, the surface is blocked with a second compound to prevent nonspecific binding of labeled assay components. Alternatively, the surface is designed such that it nonspecifically binds one component but does not significantly bind another. For example, a surface bearing a lectin such as concanavalin A will bind a carbohydrate containing compound but not a labeled protein that lacks glycosylation. Various solid surfaces for use in noncovalent attachment of assay components are reviewed in U.S. Pat. Nos. 4,447,576 and 4,254,082.

D) Other Assay Formats

BoNT/A polypeptides or BoNT/A-neutralizing antibodies can also be detected and quantified by any of a number of other means well known to those of skill in the art. These include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitin reactions, immunodiffusion (single or double), immunoelectrophoresis, radioimmunoassays (RIAs), enzyme-linked immunosorbent assays (ELISAs), immunofluorescent assays, and the like.

Western blot analysis and related methods can also be used to detect and quantify the presence of BoNT/A polypeptides in a sample. The technique generally comprises separating sample products by gel electrophoresis on the basis of molecular weight, transferring the separated products to a suitable solid support, (such as a nitrocellulose filter, a nylon filter, or derivatized nylon filter), and incubating the sample with the antibodies that specifically bind either the BoNT/A polypeptide. The antibodies specifically bind to the biological agent of interest on the solid support. These antibodies are directly labeled or alternatively are subsequently detected using labeled antibodies (e.g., labeled sheep anti-human antibodies where the antibody to a marker gene is a human antibody) which specifically bind to the antibody which binds BoNT/A.

Other assay formats include liposome immunoassays (LIAs), which use liposomes designed to bind specific molecules (e.g., antibodies) and release encapsulated reagents or markers. The released chemicals are then detected according to standard techniques (see, Monroe et al., (1986) Amer. Clin. Prod. Rev. 5:34-41).

E) Labeling of BoNT/A-neutralizing Antibodies

The labeling agent can be, e.g., a monoclonal antibody, a polyclonal antibody, a protein or complex such as those described herein, or a polymer such as an affinity matrix, carbohydrate or lipid. Detection proceeds by any known method, including immunoblotting, western analysis, gel-mobility shift assays, tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. The particular label or detectable group used in the assay is not a critical aspect of the invention. The detectable group can be any material having a detectable physical or chemical property. Such detectable labels have been well-developed in the field of immunoassays and, in general, any label useful in such methods can be applied to the present invention. Thus, a label is any composition detectable by spectroscopic, photochemical, biochemical, immunochemical, electrical, optical or chemical means. Useful labels in the present invention include magnetic beads (e.g. Dynabeads™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., LacZ, CAT, horse radish peroxidase, alkaline phosphatase and others, commonly used as detectable enzymes, either as marker gene products or in an ELISA), and colorimetric labels such as colloidal gold or colored glass or plastic (e.g. polystyrene, polypropylene, latex, etc.) beads.

The label may be coupled directly or indirectly to the desired component of the assay according to methods well known in the art. As indicated above, a wide variety of labels may be used, with the choice of label depending on the sensitivity required, ease of conjugation of the compound, stability requirements, available instrumentation, and disposal provisions.

Non radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems which may be used, see, U.S. Pat. No. 4,391,904, which is incorporated herein by reference.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence, e.g., by microscopy, visual inspection, via photographic film, by the use of electronic detectors such as charge coupled devices (CCDs) or photomultipliers and the like. Similarly, enzymatic labels may be detected by providing appropriate substrates for the enzyme and detecting the resulting reaction product. Finally, simple calorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of BoNT/A peptides. In this case, antigen-coated particles are agglutinated by samples comprising the target antibodies. In this format, none of the components need be labeled and the presence of the target antibody is detected by simple visual inspection.

V. Pharmaceutical Compositions

The BoNT/A-neutralizing antibodies of this invention are useful for parenteral, topical, oral, or local administration, such as by aerosol or transdermally, for prophylactic and/or therapeutic treatment. The pharmaceutical compositions can be administered in a variety of unit dosage forms depending upon the method of administration. For example, unit dosage forms suitable for oral administration include powder, tablets, pills, capsules and lozenges. It is recognized that the fusion proteins and pharmaceutical compositions of this invention, when administered orally, must be protected from digestion. This is typically accomplished either by complexing the protein with a composition to render it resistant to acidic and enzymatic hydrolysis or by packaging the protein in an appropriately resistant carrier such as a liposome. Means of protecting proteins from digestion are well known in the art.

The pharmaceutical compositions of this invention are particularly useful for parenteral administration, such as intravenous administration or administration into a body cavity or lumen of an organ. The compositions for administration will commonly comprise a solution of the BoNT/A-neutralizing antibody dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of BoNT/A-neutralizing antibody in these formulations can vary widely, and will be selected primarily based on fluid volumes, viscosities, body weight and the like in accordance with the particular mode of administration selected and the patient's needs.

Thus, a typical pharmaceutical composition for intravenous administration would be about 0.1 to 10 mg per patient per day. Dosages from 0.1 up to about 100 mg per patient per day may be used, particularly when the drug is administered to a secluded site and not into the blood stream, such as into a body cavity or into a lumen of an organ. Methods for preparing parenterally administrable compositions will be known or apparent to those skilled in the art and are described in more detail in such publications as Remington's Pharmaceutical Science, 15th ed., Mack Publishing Company, Easton, Pa. (1980).

The compositions containing the present fusion proteins or a cocktail thereof (i.e., with other proteins) can be administered for therapeutic treatments. In therapeutic applications, Preferred pharmaceutical compositions are administered in a dosage sufficient to neutralize (mitigate or eliminate) BoNT/A toxin (i.e., reduce or eliminate a symptom of BoNT/A poisoning (botulism)). An amount adequate to accomplish this is defined as a “therapeutically effective dose.” Amounts effective for this use will depend upon the severity of the disease and the general state of the patient's health.

Single or multiple administrations of the compositions may be administered depending on the dosage and frequency as required and tolerated by the patient. In any event, the composition should provide a sufficient quantity of the proteins of this invention to effectively treat the patient.

VI. Kits For Diagnosis or Treatment

In another embodiment, this invention provides for kits for the treatment of botulism or for the detection/confirmation of a Clostridium botulinum infection. Kits will typically comprise one or more BoNT/A-neutralizing antibodies of this invention. For diagnostic purposes, the antibody(s) can be labeled. In addition the kits will typically include instructional materials disclosing means of use BoNT/A-neutralizing antibodies in the treatment of symptoms of botulism. The kits may also include additional components to facilitate the particular application for which the kit is designed. Thus, for example, where a kit contains a BoNT/A-neutralizing-antibody antibody is labeled, , the kit may additionally contain means of detecting the label (e.g. enzyme substrates for enzymatic labels, filter sets to detect fluorescent labels, appropriate secondary labels such as a sheep anti-human antibodies, or the like). The kits may additionally include buffers and other reagents routinely used for the practice of a particular method. Such kits and appropriate contents are well known to those of skill in the art.

### EXAMPLES

The following examples are provided by way of illustration only and not by way of limitation. Those of skill will readily recognize a variety of noncritical parameters that can be changed or modified to yield essentially similar results.

### Example 1

### Preparation of Botulinum Neurotoxin Neutralizing Antibodies

Materials and Methods

A) Oligonucleotide Design

Family-specific murine V_{H} and V_{K} primers were designed as previously described for human V-gene primers (Marks, et al. (1991) J. Mol. Biol. 222:581-597; Marks, et al., Eur. J. Immunol. 21:985-991) to amplify full-length rearranged V genes. Briefly, murine V_{H} and V_{K} DNA sequences were collected from the Kabat (Kabat, et al. (1991) Sequences of proteins of immunological interest, U.S. Department of Health and Human Services, U.S. Government Printing Office, Bethesda, Md.) and GenBank databases, aligned, and classified by family, and family-specific primers were designed to anneal to the first 23 nucleotides comprising framework 1. Similarly, J_{H} and J_{K} gene-segment specific primers were designed to anneal to the final 24 nucleotides comprising each of the 4 J_{H} and 5 J_{K} gene segments (Kabat, et al. supra.).

B) Vector Construction

To construct the vector pSYN3, a 1.5 kb stuffer fragment was amplified from pCANTAB5E (Pharmacia Biotech, Milwaukee, Wis.) using PCR with the primers LMB3 (Marks, et al. (1991) Eur. J. Immunol. 21:985-991) and E-tagback (5′-ACC ACC GAA TTC TTA TTA ATG GTG ATG ATG GTG GAT GAC CAG CCG GTT CCA GCG G-3′, SEQ ID NO: ______). The DNA fragment was digested with SfiI and Notf, gel purified, and ligated into pCANTAB5E digested with SfiI and NotI. Ligated DNA was used to transform Escherichia coli TG1 (Gibson (1991) Studies on the Epstein-Barr virus genome. University of Cambridge, Cambridge, U.K.), and clones containing the correct insert were identified by DNA sequencing. The resulting vector permits subcloning of phage-displayed scFv as SfiI-NotI or Mcol-NotI fragments for secretion into the periplasm of E. coli as native scFv with a C-terminal E epitope tag followed by a hexahistidine tag.

C) Immunizations

For construction of library 1, BALB/c mice (16 to 22 g) were immunized at 0, 2, and 4 weeks with pure BoNT/A H_{c} (Ophidian Pharmaceuticals, Madison, Wis.). Each animal was given subcutaneously 1 μg of material adsorbed onto alum (Pierce Chemical Co., Rockford, Ill.) in a volume of 0.5 ml. Mice were challenged 2 weeks after the second immunization with 100,000 50% lethal doses of pure BoNT/A and were sacrificed 1 week later.

For construction of library 2, CD-1 mice (16 to 22 g) were immunized at 0, 2, and 4 weeks with pure BoNT/A H_{c} and were sacrificed two weeks after the third immunization. For both libraries, the spleens were removed immediately after sacrifice and total RNA was extracted by the method of Cathala et al. (1993) DNA 2: 329.

D) Library Construction

First-strand cDNA was synthesized from approximately 10 μg of total RNA as previously described in Marks, et al. (1991) J. Mol. Biol. 222:581-597, except that immunoglobulin mRNA was specifically primed with 10 pmol each of oligonucleotides MIgG1 For, MIgG3 For, and MC_{K} For (Table 1). For construction of library 1, rearranged V_{H}, and V_{K} genes were amplified from first-strand cDNA by using commercially available V_{H} and V_{K} back primers and J_{H} and J_{K} forward primers (Recombinant Phage Antibody System; Pharmacia Biotech). For library 2, equimolar mixtures of family-specific V_{H} and V_{K} back primers were used in conjunction with equimolar mixtures of J_{H} or J_{K} gene-segment-specific forward primers in an attempt to increase library diversity (see “Oligonucleotide design” above). Re-arranged V_{H} and V_{K} genes were amplified separately in 50-μl reaction mixtures containing 5 μl of the first-strand CDNA reaction mixture, 20 pmol of an equimolar mixture of the appropriate back primers, 20 pmol of an equimolar mixture of the appropriate forward primers, 250 μm (each) deoxynucleoside triphosphate, 1.5 mm MgCI_{2, 10} μg of bovine serum albumin/ml, and 1 μl (5 U) of Thermus aquaticus (Taq) DNA polymerase (Promega) in the buffer supplied by the manufacturer. The reaction mixture was overlaid with paraffin oil (Sigma) and cycled 30 times (at 95° C. for 1 min, 60° C. for 1 min, and 72° C. for 1 min). Reaction products were gel purified, isolated from the gel by using DEAE membranes, eluted from the membranes with high-salt buffer, ethanol precipitated, and resuspended in 20 μL of water (Sambrook, et al. (1989) Molecular cloning; a laboratory manual, 2nd ed. Cold Spring Harbor Laboratory, Cold Spring Harbor, N.Y.).

TABLE 1
Oligonucleotide primers used for PCR of mouse immunoglobulin genes.

| | |
|---|---|
| Primer ID | Sequence |

A. 1st strand cDNA synthesis
Mouse heavy chain constant region primers

| | |
|---|---|
| MIgG1/2 For | 5′ CTG GAC AGG GAT CCA GAG TTC CA 3′ |
| MIgG3 For | 5′ CTG GAC AGG GCT CCA TAG TTC CA 3′ |

Mouse constant region primer

| | |
|---|---|
| MC_{K} For | 5′ CTC ATT CCT GTT GAA GCT CTT GAC 3′ |

B. Primary PCR
Mouse V_{H} back primers

| | |
|---|---|
| V_{H}1 Back | 5′ GAG GTG CAG CTT CAG GAG TCA GG 3′ |
| V_{H}2 Back | 5′ GAT GTG CAG CTT CAG GAG TCR GG 3′ |
| V_{H}3 Back | 5′ CAG GTG CAG CTG AAG SAG TCA GG 3′ |
| V_{H}4/6 Back | 5′ GAG GTY CAG CTG CAR CAR TCT GG 3′ |
| V_{H}5/9 Back | 5′ CAG GTY CAR CTG CAG CAG YCT GG 3′ |
| V_{H}7 Back | 5′ GAR GTG AAG CTG GTG GAR TCT GG 3′ |
| V_{H}8 Back | 5′ GAG GTT CAG CTT CAG CAG TCT GG 3′ |
| V_{H}10 Back | 5′ GAA GTG CAG CTG KTG GAG WCT GG 3′ |
| V_{H}11 Back | 5′ CAG ATC CAG TTG CTG CAG TCT GG 3′ |

Mouse V_{H} back primers

| | |
|---|---|
| V_{H}1 Back | 5′ GAC ATT GTG ATG WCA CAG TCT CC 3′ |
| V_{H}2 Back | 5′ GAT GTT KTG ATG ACC CAA ACT CC 3′ |
| V_{H}3 Back | 5′ GAT ATT GTG ATR ACB CAG GCW GC 3′ |
| V_{H}4 Back | 5′ GAC ATT GTG CTG ACM CAR TCT CC 3′ |
| V_{H}5 Back | 5′ SAA AWT GTK CTC ACC CAG TCT CC 3′ |
| V_{H}6 Back | 5′ GAY ATY VWG ATG ACM CAG WCT CC 3′ |
| V_{H}7 Back | 5′ CAA ATT GTT CTC ACC CAG TCT CC 3′ |
| V_{H}8 Back | 5′ TCA TTA TTG CAG GTG CTT GTG GG 3′ |

Mouse J_{H} forward primers

| | |
|---|---|
| J_{H}1 For | 5′ TGA GGA GAC GGT GAC CGT GGT CCC 3′ |
| J_{H}2 For | 5′ TGA GGA GAC TGT GAG AGT GGT GCC 3′ |
| J_{H}3 For | 5′ TGC AGA GAC AGT GAC CAG AGT CCC 3′ |
| J_{H}4 For | 5′ TGA GGA GAC GGT GAC TGA GGT TCC 3′ |

Mouse J_{K} forward primers

| | |
|---|---|
| J_{K}1 For | 5′ TTT GAT TTC CAG CTT GGT GCC TCC 3′ |
| J_{K}2 For | 5′ TTT TAT TTC CAG CTT GGT CCC CCC 3′ |
| J_{K}3 For | 5′ TTT TAT TTC CAG TCT GGT CCC ATC 3′ |
| J_{K}4 For | 5′ TTT TAT TTC CAA CTT TGT CCC CGA 3′ |
| J_{K}5 For | 5′ TTT CAG CTC CAG CTT GGT CCC AGC 3′ |

C. Reamplification primers containing restriction sites
Mouse V_{H} Sfi back primers

| | |
|---|---|
| V_{H}1 Sfi | 5′ GTC CTC GCA ACT GCG GCC CAG CCG GCC ATG GCC GAG GTG CAG CTT CAG |
| | GAG TCA GG 3′ |
| V_{H}2 Sfi | 5′ GTC CTC GCA ACT GCG GCC CAG CCG GCC ATG GCC GAT GTG CAG CTT CAG |
| | GAG TCR GG 3′ |
| V_{H}3 Sfi | 5′ GTC CTC GCA ACT GCG GCC CAG CCG GCC ATG GCC CAG GTG CAG CTG AAG |
| | SAG TCA GG 3′ |
| V_{H}4/6 Sfi | 5′ GTC CTC GCA ACT GCG GCC CAG CCG GCC ATG GCC GAG GTY CAG CTG CAR |
| | CAR TCT GG 3′ |
| V_{H}5/9 Sfi | 5′ GTC CTC GCA ACT GCG GCC CAG CCG GCC ATG GCC CAG GTY CAR CTG CAG |
| | CAG YCT GG 3′ |
| V_{H}7 Sfi | 5′ GTC CTC GCA ACT GCG GCC CAG CCG GCC ATC GCC GAR GTG AAG CTG GTG |
| | GAR TCT GG 3′ |
| V_{H}8 Sfi | 5′ GTC CTC GCA ACT GCG GCC CAG CCG GCC ATG GCC GAG GTT CAG CTT CAG |
| | CAG TCT CG 3′ |
| V_{H}10 Sfi | 5′ GTC CTC GCA ACT GCG GCC CAG CCG GCC ATG GCC GAA GTG CAG CTG KTG |
| | GAG WCT GG 3′ |
| V_{H}11 Sfi | 5′ GTC CTC GCA ACT GCG GCC CAG CCG GCC ATC GCC CAG ATC CAG TTG CTG |
| | CAG TCT GG 3′ |

Mouse J_{K} Not forward primers

| | |
|---|---|
| J_{K}1 Not | 5′ GAG TCA TTC TCG ACT TGC GGC CGC TTT GAT TTC CAG CTT GGT GCC TCC 3′ |
| J_{K}2 Not | 5′ GAG TCA TTC TCG ACT TGC GGC CGC TTT TAT TTC CAG CTT GGT CCC CCC 3′ |
| J_{K}3 Not | 5′ GAG TCA TTC TCG ACT TGC GGC CGC TTT TAT TTC CAG TCT GGT CCC ATC 3′ |
| J_{K}4 Not | 5′ GAG TCA TTC TCG ACT TGC GGC CGC TTT TAT TTC CAA CTT TGT CCC CGA 3′ |
| J_{K}5 Not | 5′ GAG TCA TTC TCG ACT TGC GGC CGC TTT CAG CTC CAG CTT GGT CCC AGC 3′ |

R = A/G, Y = C/T, S = G/C, K = G/T, W = A/T, M = A/C, V = C/G/A, B = G/C/T, and H = C/A/T.

scFv gene repertoires were assembled from purified V_{H} and V_{K} gene repertoires and linker DNA by using splicing by overlap extension. Linker DNA encoded the peptide sequence (G₄S₃, SEQ ID NO: ______), Huston, et al. (1988) Proc. Natl. Acad. Sci. USA 85:5879-5883) and was complementary to the 3′ ends of the rearranged V_{H} genes and the 5′ ends of the rearranged V. genes. The V_{H} and V_{K} DNAs (1.5 μg of each) were combined with 500 ng of linker DNA (Recombinant Phage Antibody System; Pharmacia Biotech) in a 25 μl PCR mixture containing 250 μm (each) deoxynucteoside triphosphate, 1.5 mM MgCl, 10 μg of bovine serum albumin/ml, and 1 μl (5 U) of Taq DNA polymerase (Promega) in the buffer supplied by the manufacturer, and the mixture was cycled 10 times (at 94° C. for 1 min, 62° C. for 1 min, and 72° C. for 1 min) to join the fragments. Flanking oligonucleotide primers (RS, provided in the Recombinant Phage Antibody System kit, for library I and an equimolar mixture of V_{H}Sfi and JKNot primers [Table 1] for library 2) were added, and the reaction mixture was cycled for 33 cycles (at 94° C. for 1 min, 55° C. for 1 min, and 72° C. for 1 min) to append restriction sites.

scFv gene repertoires were gel purified as described above, digested with Sfif and Notl, and purified by electroelution, and 1 μg of each repertoire was ligated into either 1 μg of pCANTAB5E vect or (Pharmacia Biotech) (library 1) or 1 μg of pHEN-1 (Hoogenboom, et al. (1991) Nucleic Acids Res. 19: 4133-4137) (library 2) digested with Sfil and Notl. The ligation mix was purified by extraction with phenol-chloroform, ethanol precipitated, resuspended in 20 μl of water, and 2.5 μl samples were electroporated (Dower, et al. (1988) Nucleic Acids Res. 16:6127-6145) into 50 μl of E. coli TGI (Gibson (1984), Studies on the Epstein-Barr virus genome. University of Cambridge, Cambridge, U.K.). Cells were grown in 1 ml of SOC (Sambrook, et al.supra.) for 30 min and then plated on TYE (Miller (1972) Experiments in molecular genetics., Cold Spring Harbor Laboratory, Cold Spring Harbor, N.Y.) medium containing 100 μg of AMP/ml and 1% (wt/vol) GLU(TYE-AMP-GLU). Colonies were scraped off the plates into 5 ml of 2×TY broth (Miller (1972) supra.) containing 100 μg of AMP/ml, 1% GLU (2×TY-AMP-GLU), and 15% (vol/vol) glycerol for storage at −70° C. The cloning efficiency and diversity of the libraries were determined by PCR screening (Gussow, et al. (1989), Nucleic Acids Res. 17: 4000) as described by Marks et al. (1991) Eur. J. Immunol., 21: 985-991.

E) Preparation of Phage

To rescue phagemid particles from the libraries, 10 ml of 2×. TY-AMP-GLU was inoculated with an appropriate volume of bacteria (approximately 50 to 100 μl) from the library stocks to give an A₆₀₀ of 0.3 to 0.5 and bacteria were grown for 30 min with shaking at 37° C. About 10¹² PFU of VCS-M13 (Stratagene) particles were added, and the mixture was incubated at overnight at 4° C. Tubes were blocked for 1 h at 37° C. with 2% MPBS, and selection, washing, and elution were performed exactly as described in reference 35 by using phage at a concentration of 5.0×10¹² TU/ml. One-third of the eluted phage was used to infect 10 ml of log-phase E. coli TGI, which was plated on TYE-AMP-GLU plates as described above.

The rescue-selection-plating cycle was repeated three times, after which clones were analyzed for binding by ELISA. Libraries were also selected on soluble BoNT/A H_{c}. For library 1, 1.0 mg of BoNT/A H_{c} (700 μg/ml) was biotinylated (Recombinant Phage Selection Module; Pharmacia) and purified as recommended by the manufacturer. For each round of selection, 1 ml of phage (approximately 10¹³ TU) were mixed with 1 ml of PBS containing 4% skim milk powder, 0.05% Tween 20, and 10 μg of biotinylated BoNT/A H_{c}/ml. After 1 h at room temperature, antigen-bound phage were captured on blocked streptavidin-coated M280 magnetic beads (Dynabeads; Dynal) as described by Schier et al. (1996) J. Mol. Biol., 255: 28-43. Dynabeads were washed a total of 10 times (three times in TPBS, twice in TMPBS, twice in PBS, once in MPBS, and two more times in PBS). Bound phage were eluted from the Dynabeads by incubation with 100 μl of 100 mM triethylamine for 5 min and were neutralized with 1 M Tris-HCl, pH 7.5, and one-third of the eluate was used to infect log-phase E. coli TG1.

For library 2, affinity-driven selections (Hawkins, et al. (1992) J. Mol. Biol. 226: 889-896; Schier, et al. (1996) supra.)) were performed by decreasing the concentration of soluble BoNT/A H_{c} used for selection (10 μg/ml for round 1, 1 μg/ml for round 2, and 10 ng/ml for round 3). Soluble BoNT/A H_{c} was captured on 200 μl of Ni²⁺-NTA (Qiagen) via a C-terminal hexahistidine tag. After capture, the Ni²⁺-NTA resin was washed a total of 10 times (5 times in TPBS and 5 times in PBS), bound phage were eluted as described above, and the eluate was used to infect log-phase E. coli TGI.

F) Initial Characterization of Binders

Initial analysis for binding to BoNT/A, BoNT/A H_{c}, and BoNT/A H_{N} (Chen, et al. (1997) Infect. Immun. 65: 1626-1630) was performed by ELISA using bacterial supernatant containing expressed scFv. Expression of scFv (De Bellis, et al., (1990) Nucleic Acids Res. 18: 1311) was performed in 96-well microtiter plates as described by marks et al. (1991) J. Mol. Biol., 222: 581-597. For ELISA, microtiter plates (Falcon 3912) were coated overnight at 4° C. with either BoNT/A, BoNT/A H_{c}, or BoNT/A H_{N} (10 μg/ml) in PBS and then were blocked with 2% MPBS for 1 h at room temperature. Bacterial supernatants containing expressed scFv were added to wells and incubated at room temperature for 1.5 h. Plates were washed six times (3 times with TPBS and 3 times with PBS), and binding of scFv was detected via their C-terminal peptide tags (E epitope tag for library 1 in pCANTAB5E and myc epitope tag [Munro, et al. (1986) Cell 46: 291-300] for library 2 in pHEN-1) by using either anti-myc tag antibody (9E10; Santa Cruz Biotechnology) or anti-E antibody (Pharmacia Biotech) and peroxidase-conjugated anti-mouse Fc antibody (Sigma), as described by Marks et al. (1991) J. Mol. Biol., 222: 581-597 and Schier et al. (1996) Gene 169: 147-155. The number of unique binding scFv was determined by BstN1 fingerprinting and DNA sequencing.

G Subcloning, Expression, and Purification of scFv

To facilitate, purification, scFv genes were subcloned into the expression vector pUC119mycHis (Schier et al. (1995) J. Mol. Biol., 263: 551-567) or pSYN3, resulting in the addition of a hexahistidine tag at the C-terminal end of the scFv. Two hundred-milliliter cultures of E. coli TG1 harboring one of the appropriate phagemids were grown, expression of scFv was induced with IPTG (De Bellis, et al. (1990), Nucleic Acids Res. 18:1311), and the cultures were grown at 25° C. overnight. scFv was harvested from the periplasm (Breitling, et al. (1991) Gene 104:147-153), dialyzed overnight at 4° C. against IMAC loading buffer (50 mM sodium phosphate [pH 7.5], 500 mM NaCl, 20 mM imidazole), and then filtered through a 0.2-μm-pore-size filter. scFv was purified by IMAC (Hochuli, et al. (1988) Bio/Technology 6: 1321-1325) as described by Schier et al. (1995) supra.

To separate monomeric scFv from dimeric and aggregated scFv, samples were concentrated to a volume of <1 ml in a centrifugal concentrator (Centricon 10; Amicon) and fractionated on a Superdex 75 column (Pharmacia) by using HBS. The purity of the final preparation was evaluated by assaying an aliquot by sodium dodecyl sulfate-polyacrylamide gel electrophoresis. Protein bands were detected by Coomassie blue staining. The concentration was determined spectrophotometrically, on the assumption that an A₂₈₀ of 1.0 corresponds to an scFv concentration of 0.7 mg/ml.

H) Measurement of Affinity and Binding Kinetics

The K_{d}s of purified scFv were determined by using surface plasmon resonance in a BIAcore (Pharmacia Biosensor AB). In a BIAcore flow cell, approximately 600 RU of BoNT/A H_{c} (15 μg/ml in 10 mM sodium acetate [pH 4.5]) was coupled to a CM5 sensor chip by using N-hydroxysuccininide-N-ethyl-N′-(dimethylaminopropyl) carbodimide chemistry (Johnson, et al. (1991) Anal. Biochem. 198: 268-277). This amount of coupled BoNT/A H_{c} resulted in a maximum RU of 100 to 175 of scFv bound. For regeneration of the surface after binding of scFv, 5 μl of 4 M MgCl₂ was injected, resulting in a return to baseline. The surface was reused 20 to 30 times under these regeneration conditions. Association was measured under a continuous flow of 5 μl/min with a concentration range from 50 to 1,000 nM. kₒₙ was determined from a plot of ln (dR/dt)/t versus concentration, where R is response and t is time (Karlsson, et al. (1991) J. Immunol. Methods 145: 229-240). k_{off} was determined from the dissociation part of the sensorgram at the highest concentration of scFv analyzed (Karlsson, et al. (1991) J. Immunol. Methods 145: 229-240) by using a flow rate of 30 μl/min. K_{d} was calculated as k_{off}/kₒₙ.

I) Epitope Mapping

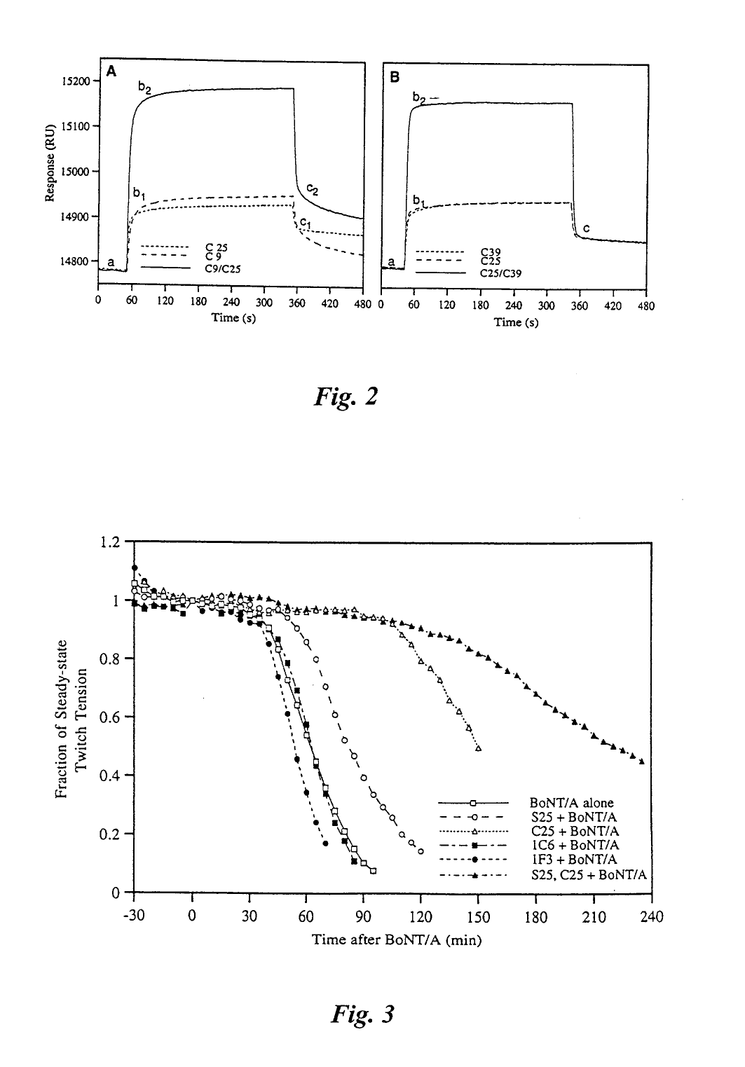

Epitope mapping was performed by using surface plasmon resonance in a BIAcore. In a BlAcore flow cell, approximately 1,200 RU of BoNT/A H_{c} was coupled to a CM5 sensor chip as described above. With a flow rate of 5 μl/min, a titration of 100 nM to 1 μM scFv was injected over the flow cell surface for 5 min to determine an scFv concentration which resulted in near saturation of the surface. Epitope mapping was performed with pairs of scFv at concentrations resulting in near saturation and at least 100 RU of scFv bound. The amount of scFv bound was determined for each member of a pair, and then the two scFv were mixed together to give a final concentration equal to the concentration used for measurements of the individual scFv. scFv recognizing different epitopes showed an additive increase in the RU bound when injected together (FIG. 2 panel A), while scFv recognizing identical epitopes showed only a minimal increase in RU (FIG. 2 panel B).

J) In vitro Neutralization Studies

In vitro neutralization studies were performed by using a mouse hemidiaphragm preparation, as described by Deshpande et al. (1995) Toxicon 33: 551-557. Briefly, left and right phrenic nerve hemidiaphragm preparations were excised from male CD/1 mice (25 to 33 g) and suspended in physiological solution (135 mM NaCl, 5 mM KCl 15 mM NaHCO₃, 1 mM Na2HPO₄, 1 mM MgCl₂, 2 mM CaCl₂, and 11 mM GLU). The incubation bath was bubbled with 95% O₂-5% CO₂ and maintained at a constant temperature of 36° C. Phrenic nerves were stimulated supramaximally at 0.05 Hz with square waves of 0.2 ms duration. Isometric twitch tension was measured with a force displacement transducer (Model FT03; Grass) connected to a chart recorder. Purified scFv were incubated with purified BoNT/A for 30 min at room temperature and then added to the tissue bath, resulting in a final scFv concentration of 2.0×10⁻⁸ M and a final BoNT/A concentration of 2.0×10⁻¹¹ M. For each scFv studied, time to 50% twitch tension reduction was determined three times for BoNT/A alone and three times for scFv plus BoNT/A. The combination of S25 and C25 was studied at a final concentration of 2.0×10⁻⁸ M each. Differences between times to 50% twitch reduction were determined by a two-tailed t test, with a P value of <0.05 considered significant.

TABLE 2
Frequency of binding of clones from phage antibody libraries

| | Frequency of ELISA-positive | | |
|---|---|---|---|
| | clones^{a} in selection round: | | |
| Antigen used for selection | 1 | 2 | 3 |
| Library 1^{b} | | | |
| BoNT/A: immunotube^{c} | 20/184 | 124/184 | ND |
| BoNT/A H_{c}: immunotube | 7/92 | 86/92 | 88/92 |
| BoNT/A H_{c}: biotinylated^{d} | 7/90 | 90/90 | 90/90 |
| | 14/48 | 48/48 | ND |
| Library 2^{e} | | | |
| BoNT/A: immunotube | ND | 81/92 | ND |
| BoNT/A H_{c}: immunotube | ND | ND | 76/92 |
| BoNT/A H,: Ni²⁺-NTA^{f} | ND | ND | 67/92 |

^{a}Expressed as number of positive clones/total number of clones. For selections on BoNT/A and BoNT/A H_{c}, ELISA was done on immobilized BoNT/A and BoNT/A H_{c}, respectively.
ND, data not determined from selection performed.
^{b}Derived from a mouse immunized twice with BoNT/A H_{c} and once with BoNT/A.
^{c}Immunotube selections were performed with the antigen absorbed onto immunotubes.
^{d}Biotinylated selections were performed in solution with capture on streptavidin magnetic beads.
^{e}Defived from a mouse immunized three times with BoNT/A H_{c}.
^{f}Ni²⁺-NTA selections were performed in solution with capture on Ni²⁺-NTA agarose.

Results

A) Phage Antibody Library Construction and Characterization

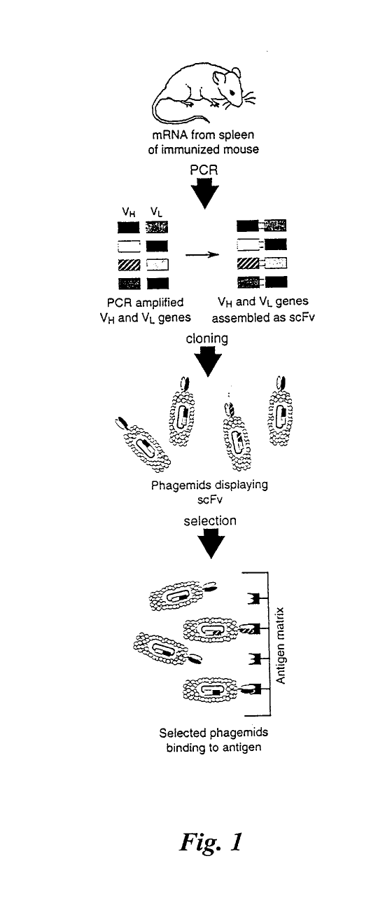

Two phage antibody libraries were constructed from the V_{H} and V_{K} genes of immunized mice (FIG. 1). For library 1, a mouse was immunized twice with BoNT/A H_{C} and challenged 2 weeks after the second immunization with 100,000 50% lethal doses of BoNT/A. The mouse survived the BoNT/A challenge and was sacrificed 1 week later. The spleen was removed immediately after sacrifice, and total RNA was prepared. For library construction, IgG heavy-chain and kappa light-chain mRNA were specifically primed and first-strand cDNA was synthesized. V_{H} and V_{K} gene repertoires were amplified by PCR, and V_{H}, J_{H} V_{K}, and J_{K} primers were provided in the recombinant phage antibody system.

The V_{H} and V_{K} gene repertoires were randomly spliced together to create an scFv gene repertoire by using synthetic DNA encoding the 15-amino-acid peptide linker (G₄S)₃. Each scFv gene repertoire was separately cloned into the phage display vector pCANTAB5E (Pharmacia). After transformation, a library of 2.1×10⁶ members was obtained. Ninety percent of the clones had an insert of the appropriate size for an scFv gene, as determined by PCR screening, and the cloned scFv genes were diverse, as determined by PCR fingerprinting. DNA sequencing of 10 unselected clones from library 1 revealed that all V_{H} genes were derived from the murine V_{H} 2 family and all V_{K} genes were derived from the murine V_{K} 4 and V_{K} 6 families (Kabat, et al. (1991) supra.). Based on this observed V-gene bias, family-specific V_{H} and V_{K} primers were designed along with J_{H} and J_{K} gene-segment-specific primers (Table 1). These primers were then used to construct a second phage antibody library.

For library 2, a mouse was immunized three times with BoNT/A H_{c} and sacrificed 2 weeks after the third immunization. The mouse was not challenged with BoNT/A prior to spleen harvest, as this led to the production of non-H_{c}-binding antibodies (see “Selection and initial characterization of phage antibodies” below). The spleen was harvested, and a phage antibody library was constructed as described above, except that V_{H}-, J_{H}-, V_{K}-, and J_{K}-specific primers were used. After transformation, a library of 1.0×10⁶ members was obtained. Ninety-five percent of the clones had an insert of the appropriate size for an scFv gene, as determined by PCR screening, and the cloned scFv genes were diverse, as determined by PCR fingerprinting (data not shown). DNA sequencing of 10 unselected clones from library 2 revealed greater diversity than was observed in library 1; V_{H} genes were derived from the V_{H1}, V_{K}2, and V_{K}3 families, and V_{K} genes were derived from the V_{K}2, V_{K}3, V_{K}4, and V_{K}6 families (Kabat, et al. (1991) supra.).

B) Selection and Initial Characterization of Phage Antibodies

To isolate BoNT/A binding phage antibodies, phage were rescued from the library and selected on either purified BoNT/A or BoNT/A H_{c}. Selections were performed on the holotoxin in addition to H_{c}, since it was unclear to what extent the recombinant toxin H_{c} would mimic the conformation of the H_{c} in the holotoxin. Selection for BoNT/A and BoNT/A H_{c} binders was performed on antigen adsorbed to polystyrene. In addition, H_{c} binding phage were selected in solution on biotinylated H_{c}, with capture on streptavidin magnetic beads (for library 1) or on hexahistidine tagged H_{c}, with capture on Ni²⁺-NTA agarose (for library 2). Selections in solution were utilized based on our previous observation that selection on protein adsorbed to polystyrene could yield phage antibodies that did not recognize native protein (Schier et al. (1995) Immunotechnology, 1: 73-81). Selection in solution was not performed on the holotoxin due to our inability to successfully biotinylate the toxin without destroying immunoreactivity.

After two to three rounds of selection, at least 67% of scFv analyzed bound the antigen used for selection (Table 2). The number of unique scFv was determined by DNA fingerprinting followed by DNA sequencing, and the specificity of each scFv was determined by ELISA on pure BoNT/A and recombinant BoNT/A H_{c} and HN scFv binding BoNT/A but not binding, H_{c} or HN were presumed to bind the light chain (catalytic domain). A total of 33 unique scFv were isolated from mice immunized with H_{c} and challenged with BoNT/A (Table 3, library 1). When library 1 was selected on holotoxin, 25 unique scFv were identified. Only 2 of these scFv, however, bound H_{c}, with the majority (Hathaway, et al. (1984) J. Infect. Dis. 150:407-412) binding the light chain and 2 binding H_{N}. The two H_{c} binding scFv did not express as well as other scFv recognizing similar epitopes, and they were therefore not characterized with respect to affinity or neutralization capacity (see below).

Selection of library 1 on H_{c} yielded an additional eight unique scFv (Tables 3 and 4). Overall, however, only 50% of scFv selected on H_{c} also bound holotoxin. This result suggests that a significant portion of the H_{c} surface may be inaccessible in the holotoxin. Alternatively, scFv could be binding, H_{c} conformations that do not exist in the holotoxin. From mice immunized with H_{c} only (library 2), all scFv selected on holotoxin also bound H_{c}. As with library 1, however, only 50% of scFv selected on H_{c} bound holotoxin. In all, 18 unique H_{c} binding scFv were isolated from library 2, resulting in a total of 28 unique H_{c} binding scFv (Tables 3 and 4). scFv of identical or related sequences were isolated on both H_{c} immobilized on polystyrene and H_{c} in solution. Thus, in the case of H_{c}, the method of selection was not important.

TABLE 3
Specificity of BoNT binding scFv selected from
phage antibody libraries

Number of unique scFv

| scFv Specificity | library 1 | library 2 |
|---|---|---|
| BoNT/A H_{c} | 10 | 18 |
| BoNT/A H_{N} | 2 | 0 |
| BoNT/A light chain | 21 | 0 |
| Total | 33 | 18 |

C) Epitope Mapping

All 28 unique H_{c} binding scFv were epitope mapped using surface plasmon resonance in a BIAcore. Epitope mapping was performed with pairs of scFv at concentrations resulting in near saturation of the chip surface and at least 100 RU of scFv bound. The amount of scFv bound was determined for each member of a pair, and then the two scFv were mixed together to give a final concentration equal to the concentration used for measurements of the individual scFv. Those scFv recognizing different epitopes showed an additive increase in the RU bound when injected together (FIG. 2, panel A), while scFv recognizing identical epitopes showed only a minimal increase in RU (FIG. 2, panel B). By this technique, mapping of the 28 scFv yielded 4 nonoverlapping epitopes recognized on H_{c} (Table 4). scFv recognizing only epitopes 1 and 2 were obtained from library 1, whereas scFv recognizing all 4 epitopes were obtained from library 2.

Many of the scFv recognizing the same epitope (C1 and S25; C9 and C15; 1E8 and 1G7; 1B6 and 1C9; C25 and C39; 2G5, 3C3, 3F4, and 3H4; 1A1 and 1F1; 1B3 and 1C6; 1G5 and 1H6; 1F3 and 2E8) had V_{H} domains derived from the same V-D-J rearrangement, as evidenced by the high level of homology of the V_{H}CDR3 and V_{H}-gene segment (Table 4). These scFv differ only by substitutions introduced by somatic hypermutation or PCR error. For epitopes 1 and 2, most or all of the scFv recognizing the same epitope are derived from the same or very similar V_{H}-gene segments but differ significantly with respect to V_{H}CDR3 length and sequence (5 of 9 scFv for epitope 1; 8 of 8 scFv for epitope 2) (Table 4). These include scFv derived from different mice. Given the

TABLE 4
Deduced protein sequences of V_{H} and V_{L} of BoNT/A H_{C} binding scT_{V}, classified by epitope recognized

| Re- | Epi- | | | Sequence^{b} | | | |
|---|---|---|---|---|---|---|---|
| gion | tope | Clone | Lib^{a} | Framework 1 | CDR 1 | Framework 2 | CDR 2 |
| V_{H} | 1 | C15 | 1 | QVKLQQSGAELVRPGASVKLSCKTSGYSFT | SYWMN | WVKQGPGQGLEWIG | MIHPSNSEIRFNQKFED |
| | | C9 | 1 | ------------------------------ | ----- | -------------- | ----------------N |
| | | 1D5 | 2 | E---VE------------N----A------ | ----- | ----R--------- | --------T-L----K- |
| | | C1 | 1 | -----------------------A------ | ----- | ----R--------- | -------DT-------- |
| | | S25 | 1 | -----------------------A----L- | ----- | ----R--------- | -----D-DT-------- |
| | | 1B6 | 2 | --Q------------V---I---A---T-I | D-A-H | ----S-AKS----- | V-SSYYGDTDY--I-KG |
| | | 1C9 | 2 | --Q-K----------V---I---G---T-I | D-AVH | ----SHAKS----- | V-STYYGDADY-PK-KG |
| | | 1E8 | 2 | E-Q--E--PG--K-SQ-LS-T-TVT---I- | D-AW- | -IR-F--KK---M- | Y-S YSGSTGYNPSLKS |
| | | 1G7 | 2 | E-Q--E--PG -K-SQ-LS-T-TVT---I- | D-AWY | -IR-F--KK---M- | Y-S YSGSTGYNPSLKS |
| | 2 | 1A1 | 2 | EVKLVESGGGLVQPGGSRKLSCATSGFTFS | DYYMS | WIRQSPDKRLEWVA | TISDGGTYTYYPDSVKG |
| | | 1F1 | 2 | -----------------L-----A------ | N-G-- | -V--T--------- | M--S--S-N--S----- |
| | | C39 | 1 | Q-Q-Q-----S-K----L-----A------ | ----- | -V--T-E------- | ------S---------- |
| | | C25 | 1 | Q-Q-Q-------K----L-----A------ | ----Y | -V--T-E------- | ------S----------- |
| | | 2G5 | 2 | ------------K----L-----A------ | S-A-- | -V--T-E------- | -----------T-N--- |
| | | 3C3 | 2 | ----K-------K----L-----A------ | S-A-- | -V--T-E------- | -----------T-N--- |
| | | 3F4 | 2 | EG----------K----L-----A------ | S-A-- | -V--T-EH------ | -------F---T-N--- |
| | | 3H4 | 2 | ------------K---PL-----A------ | S-A-- | -V--T-EH------ | -------F---T-N--- |
| | 3 | 1B3 | 2 | EVQLQESGGGVVQPGRSLRLSCAASGFTFS | SYAMH | WVRQAPGKGLEWVA | VISYDGSNKYYADSVKG |
| | | 1C6 | 2 | QI--LQ------------------------ | ----- | -------------- | ----------------- |
| | | 2B6 | 2 | VKLVESGP-L-KPSQSLSLTCTVTGYSIT- | D-AWN | -I--F--NK---MG | Y-N-----N-NP -L-N |
| | | 1G5 | 2 | Q----Q--AEL----A-VKM--K---Y--T | --WTT | --K-R--Q----IG | D-YPGSGSTNYNEKF-S |
| | | 1H6 | 2 | -----Q--AEL-K--A-VKM--K---Y--T | --WTT | --K-R--Q----IG | D-YP-SGSTNYNEKF-S |
| | 4 | 1F3 | 2 | EVQLQQSGAELVKPGASVKLSCKASGYTFT | SFWMH | WVKQAPGRGLEWIG | RLDPNSGETKYNEKFKS |
| | | 2E8 | 2 | ------------------------------ | ----- | -------------- | ------------K---- |
| V_{L} | 1 | C15 | 1 | DIELTQSPAIMSASPGEKVIMTC | SASS SVSHMY | WYQQKPGSSPRLLIY | DTSNLAS |
| | | C9 | 1 | --D---------S-------I-- | ---- ---Y-H | -F-----T--KPW-- | S------ |
| | | 1D5 | 2 | -----------A--------I-- | ----S I-S-NLH | -----SETSPKPW-- | G------ |
| | | C1 | 1 | ----------------------- | ---- ---Y-- | --------------- | ------- |
| | | S25 | 1 | ---------L-A--------I-- | -V--S I-S-NLH | -----S-T--KPW-- | G------ |
| | | 1B6 | 2 | ---------SLAV-L-QRA-IS- | RA-ESVDSYGN-F-H | -------QP-K---- | RA---E- |
| | | 1C9 | 2 | ---------SLAV-L-QRA-IS- | RA-ESVDSYGN-F-H | -------QP-K---- | RA---E- |
| | | 1E8 | 2 | ----------------------- | ---- ---Y-H | -----S-T--KRW-- | ---K--- |
| | | 1G7 | 2 | ----------------------- | ---- ---Y-H | -----S-T--KRW-- | ---K--- |
| | 2 | 1A1 | 2 | DIELTQSPASLAVSLGQRATISC | RASESVDSYGNSFMH | WYQQKPGQPPKLLIY | LASNLES |
| | | 1F1 | 2 | --------T-------------- | --------------- | --------------- | ------- |
| | | C39 | 1 | ----------------R------ | ----------H---- | --------------- | ------- |
| | | C25 | 1 | ----------------------- | ----------H---Q | --------------- | R-----P |
| | | 2G5 | 2 | ---------IMSA-P-EKVTTT- | S--S SV-Y-- | -F-----TS-K-W-- | ST---A- |
| | | 3C3 | 2 | ---------IMSA-P-EKVTTT- | ----------H---Q | -F-----TS-K-W-- | ST---A- |
| | | 3F4 | 2 | -T-------IMSA-P-EKVTMT- | S--S SV-Y-Y | -------SS-R---- | DT---A- |
| | | 3H4 | 2 | ---------IMSA-P-EKVTMT- | ---S- VSS-YL- | -------SS-R---- | DT---A- |
| | 3 | 1B3 | 2 | DSELTQSPTTMAASPGEKITTTC | SASSS ISSNYLH | WYQQRPGFSPKLLIY | RTSNLAS |
| | | 1C6 | 2 | -I------ASL-V-L-RRA--S- | R--E-VEYYGTSLMQ | ----K--QP------ | AA--VE- |
| | | 2B6 | 2 | YI------ASL-V-L-QRA--S- | R--E-VDSYGNSFM- | ----K--QP------ | LA---E- |
| | | 1G5 | 2 | -I------ASL-V-L-QKA--S- | R--E-VEYYGTSLMQ | ----K--QP------ | AA--VE- |
| | | 1H6 | 2 | -I------AI-S------V---- | -V--- ---SN-- | ----KS-T----W-- | G------ |
| | 4 | 1F3 | 2 | DIELTQSPASMSASPGEKVTMTC | RATSS VSSSYLH | WYQQKSGASPKLWIY | SASNLAS |
| | | 2E8 | 2 | --------TT-A------I-I-- | S-S-- IG-N--- | -----P-F----L-- | RT----- |

| Re- | Epi- | | | Sequence^{b} | | |
|---|---|---|---|---|---|---|
| gion | tope | Clone | Lib^{a} | Framework 3 | CDR 3 | Framework 4 |
| V_{H} | 1 | C15 | 1 | MATLTVDKSSSTAYMQLSSPTSEDSAVYYCAR | GIYYDYDGGNYYAMDY | WGQGTTVTASS |
| | | C9 | 1 | ---------------------- | ----V----------- | --------V-- |
| | | 1D5 | 2 | K------------------------------- | -------E-Y--TL-- | ------L-V-- |
| | | C1 | 1 | K------R-----IH----------------- | -L-GYGF WYFDV | --------V-- |
| | | S25 | 1 | K------T------------------------ | -L-NGF WYF-V | --------V-- |
| | | 1B6 | 2 | K---------N----E-ARL--D---I----- | RGKG ---- | --------V-- |
| | | 1C9 | 2 | K-----N---N----E-PRL------I----- | RGKG ---- | -----S--V-- |
| | | 1E8 | 2 | RISI-K-T-KNQFFL--N-V-T--TGT----- | -YD ---- | -----S--V-- |
| | | 1G7 | 2 | RISI-R-T-KNQFFL--N-V-T--TGT----- | -YD ---- | -----S--V-- |
| | 2 | 1A1 | 2 | RFTISRDNAKNTLYLQMSSLKSEDTAMYYCVR | HGYGNYPSH WYFDV | WGAGTTVTVSS |
| | | 1F1 | 2 | -V--------S---------Q-------L-T- | --------Y ----- | ----------- |
| | | C39 | 1 | -----------N--------------I----- | YR-DEGL -Y | --Q-------- |
| | | C25 | 1 | -----------N------------------S- | YR-DDAM -Y | --Q-------- |
| | | 2G5 | 2 | ----------HN------H-----------A- | NLPYDHV -Y | --Q--S----- |
| | | 3C3 | 2 | ----------HN------H-----------A- | NLPYDHV -Y | --Q--S----- |
| | | 3F4 | 2 | ----------HN------H-----------A- | NLPYDHV -Y | --Q--S----- |
| | | 3H4 | 2 | ----------HN------H-----------A- | NLPYDHV -Y | --Q--S----- |
| | 3 | 1B3 | 2 | RFTISRDNSKNTLYLQMNSLRAEDTAVYYCAR | DWSEGYYYYG MDV | WGQGTTVIVSS |
| | | 1C6 | 2 | -------------------------------- | ---------- --- | ----------- |
| | | 2B6 | 2 | -ISIT--T---QFF-KL--VTS----T----- | AGDGY-VD WYFDV | --T-------- |
| | | 1G5 | 2 | KA-LTV-T-SS-A-M-LS--TS--S------- | ELGD A--Y | -----S----- |
| | | 1H6 | 2 | KA-LTV-T-SS-A-M-LS--TS--S------- | ELGD A--Y | -----S----- |
| | 4 | 1F3 | 2 | KATLTVDKPSSTAYMELSSLTSEDSAVYYCAR | EAYGYWN FDV | WGTGTTVTVSS |
| | | 2E8 | 2 | -------------------------------- | ------- --- | ----------- |
| V_{L} | 1 | C15 | 1 | GVPIRFSGSGSGTSYSLTISRMEAEDSATYYC | QQWSSYPFT | FGSGTKLELKR |
| | | C9 | 1 | ---A----------------SV----A----- | --Y-G--L- | --A-----I-- |
| | | 1D5 | 2 | ---V----------------S-----A----- | ---G---L- | --G-----I-- |
| | | C1 | 1 | ---V----------------------A----- | -------L- | --A-------- |
| | | S25 | 1 | ---V----------------S-----A----- | -------L- | --A-----I-- |
| | | 1B6 | 2 | -I-A-------R-DFT--INPV--D-V----- | --SNED-P- | --A-------- |
| | | 1C9 | 2 | -I-AK------R-DFT---NPV--D-V---- | --SNED-Y- | --G-----I-- |
| | | 1E8 | 2 | ---A----------------S-----A----- | -----N-L- | --A-------- |
| | | 1G7 | 2 | ---A----------------S-----A----- | -----N-L- | --A-------- |
| | 2 | 1A1 | 2 | GVPARFSGSGSRTDFTLTIDPVEADDAATYYC | QQNNEDPYT | QFGGGTKLEIKR |
| | | 1F1 | 2 | -------------------------------- | --------- | ----------- |
| | | C39 | 1 | -------------------------------- | --------- | ----------- |
| | | C25 | 1 | -I---------G-------N------V----- | --S----F- | --S-------- |
| | | 2G5 | 2 | -----------G-SYS---SRM--E------- | --RSSY--- | ----DQAGN-S |
| | | 3C3 | 2 | -----------G-SYS---SRM--E------- | --RSSY--- | ----DQAGN-- |
| | | 3F4 | 2 | ---V-------G-SYS---SRM--E------- | --WSSY-P- | ----------- |
| | | 3H4 | 2 | ---V-------G-SYS---SRM--E------- | --WSSY-P- | ----------- |
| | 3 | 1B3 | 2 | GVPARFSGSGSGTSYSLTIGTMEAEDVATYYC | QQGSSIPRT | FGGGTKLEIKR |
| | | 1C6 | 2 | -------------DF--N-HPV-E -I-M-F- | --SRKV-W- | ----------- |
| | | 2B6 | 2 | -----------R-DFT---DPV--D-A----- | --NNED-Y- | ----------S |
| | | 1G5 | 2 | -A-----------DF--N-HPV-ED-I-M-F- | --SRKV-Y- | ----------- |
| | | 1H6 | 2 | ---V---------------SS-----A----- | --W--Y-L- | --A---V-LR- |
| | 4 | 1F3 | 2 | GVPSRFSGSGSGTSYSLTISSVEAEDAATYYC | QQYIGYPYT | FGGGTKLEIKR |
| | | 2E8 | 2 | ---A---------------GAM----V----- | --GSSI--- | ----------- |

^{a}Lib, library.
^{b}Full-length sequences were not determined for clones C12, C13, C2, and S44 (all bind epitope 1). Accession can be made through GenBank with nos. AF003702 to af003725.

great degree of diversity in V_{H}CDR2 sequences in the primary repertoire (Tomlinson et al. (1996) J. Mol. biol., 256: 813-817), specific V_{H}-gene segments may have evolved for their ability to form binding sites capable of recognizing specific pathogenic antigenic shapes. In contrast, greater structural variation appears to occur in the rearranged Y_{K} genes. For example, three different germ line genes and CDR1 main-chain conformations (Chothia, et al. (1987) J. Mol. Biol. 196:901-917) are observed for epitope 21 where all the V_{H} (genes are derived from the same germ line gene. Such “promiscuity” in chain pairings has been reported previously (Clackson, et al. (1991) Nature 352:624-628).

D) Affinity, Binding Kinetics, and in vitro Toxin Neutralization

Affinity, binding kinetics, and in vitro toxin neutralization were determined for one representative scFv binding to each epitope. For each epitope, the scFv chosen for further study had the best combination of high expression level and slow k_{off}, as determined during epitope mapping studies. K_{d} for the four scFv studied ranged between 7.3×10⁻⁸ and 1.1×10⁻⁹ M (Table 5), values comparable to those reported for monoclonal IgG produced from hybridomas (Foote, et al., Nature 352:530-532 (1991)). C25 has the highest affinity (K_{d}=1.1×10⁻⁹ M) reported for an anti-botulinum toxin antibody. kₒₙ differed over 84-fold, and k_{off} differed over 33-fold, between scFv (Table 5). In vitro toxin neutralization was determined by using a mouse hemidiaphragm preparation and measuring the time to 50% twitch tension reduction for BoNT/A alone and in the presence of 2.0×10⁻⁸ M scFv. Values are reported in time to 50% twitch reduction. scFv binding to epitope 1 (S25) and epitope 2 (C25) significantly prolonged the time to neuroparalysis: 1.5-fold (152%) and 2.7-fold (270%), respectively (Table 5 and FIG. 3). In contrast, scFv binding to epitopes 3 and 4 had no significant effect on the time to neuroparalysis. A mixture of S25 and C25 had a significant additive effect on the time to neuroparalysis, with the time to 50% twitch reduction increasing 3.9-fold (390%).

TABLE 5
Affinities, binding kinetics, and in vitro toxin neutralization results of scFv
selected from phage antibody libraries

| scFv | | K_{d}^{a} | kₒₙ | k_{off} | |
|---|---|---|---|---|---|
| clone | Epitope | (M) | (10⁴ M⁻¹ s⁻¹) | (10⁻³ s⁻¹) | Paralysis Time^{b} |
| S25 | 1 | 7.3 × 10⁻⁸ | 1.1 | 0.82 | 85 ± 10^{c} |
| C25 | 2 | 1.1 × 10⁻⁹ | 30 | 0.33 | 151 ± 12^{c} |
| C39 | 2 | 2.3 × 10⁻⁹ | 14 | 0.32 | 139 ± 8.9^{c} |
| 1C6 | 3 | 2.0 × 10⁻⁸ | 13 | 2.5 | 63 ± 3.3 |
| 1F3 | 4 | 1.2 × 10⁻⁸ | 92 | 11 | 52 ± 1.4 |
| C25 + S25 Combination | | | | | 218 ± 22^{c, d} |
| BoNT/A pure toxin | | | | | 56 ± 3.8 |
| (control) | | | | | |

^{a} kₒₙ and k_{off} were measured by surface plasmon resonance and K_{d} calculated as k_{off}/kₒₙ.
^{b} Time (min.) to 50% twitch reduction in mouse hemidiaphragm assay using 20 nM scFv + 20 pM BoNT/A, compared to time for BoNT/A alone. For C25 + S25 combination, 20 nM scFv each was used. Each value is the mean ± SEM of at least three observations.
^{c} p < 0.01 compared to BoNT/A.
^{d} p < 0.05 compared to C25

Discussion

BoNTs consist of a heavy and a light chain linked by a single disulfide bond. The carboxy-terminal half of the toxin binds to a specific membrane receptor(s), resulting in internalization, while the amino-terminal half mediates translocation of the toxin from the endosome into the cytosol. The light chain is a zinc endopeptidase which cleaves an essential synaptosomal protein, leading to failure of synaptic transmission and paralysis. Effective immunotherapy must prevent binding of the toxin to the receptor, since the other two toxin functions occur intracellularly. Identification of epitopes on H_{c} which mediate binding is an essential first step, both to the design of better vaccines and to development of a high-titer neutralizing monoclonal antibody (or antibodies) for passive immunotherapy.

For this work, we attempted to direct the immune response to a neutralizing epitope(s) by immunization with recombinant BoNT/A H_{c}. This should lead to the production of antibodies which prevent binding of toxin to its cellular receptor(s). One limitation of this approach is the extent to which recombinant H_{c} mimics the conformation of H_{c} in the holotoxin. The fact that 50% of antibodies selected on H_{c} recognize holotoxin suggests significant structural homology for a large portion of the molecule. Although 50% of antibodies selected on H_{c} do not bind holotoxin, this could result from packing of a significant portion of the H_{c} surface against other toxin domains. Our results do not, however, exclude the possibility that some of these antibodies are binding H_{c} conformations that do not exist in the holotoxin or that conformational epitopes present in the holotoxin are absent from recombinant H_{c}. This could lead to failure to generate antibodies to certain conformational epitopes. Regardless, immunizing and selecting with H_{c} resulted in the isolation of a large panel of monoclonal antibodies which bind holotoxin. In contrast, monoclonal antibodies isolated after immunization with holotoxin or toxoid bind to other toxin domains (H_{N} or light chain) or to nontoxin proteins present in crude toxin preparations and toxoid (see results from library 1, above, and Emanuel et al. (1996)j. Immunol. Meth., 193: 189-197).

To produce and characterize the greatest number of monoclonal antibodies possible, we used phage display. This approach makes it possible to create and screen millions of different antibodies for binding. The resulting antibody fragments are already cloned and can easily be sequenced to identify the number of unique antibodies. Expression levels in E. coli are typically adequate to produce milligram quantities of scFv, which can easily be purified by IMAC after subcloning into a vector which attaches a hexahistidine tag to the C terminus. Ultimately, the V_{H} and V_{L} genes can be subcloned to construct complete IcG molecules, grafted to construct humanized antibodies, or mutated to create ultrahigh-affinity antibodies. By this approach, 28 unique monoclonal anti-BoNT/A H_{c} antibodies were produced and characterized.

The antibody sequences were diverse, consisting of 3 different V_{H}-gene families, at least 13 unique V-D-J. rearrangements, and 3 V_{K}-gene families. Generation of this large panel of BoNT/A H_{c} antibodies was a result of the choice of antigen used for immunization and selection (BoNT/A H_{c}). For example, a Fab phage antibody library constructed from the V genes of mice immunized with pentavalent toxoid yielded only two Fab which bound pure toxin (in this case, BoNT/B). The majority of the Fab bound nontoxin proteins present in the toxoid (Emanuel, et al., J. Immunol. Methods 193:189-197 (1996)).

Despite the sequence diversity of the antibodies, epitope mapping revealed only four nonoverlapping epitopes. Epitopes 1 and 2 were immunodominant, being recognized by 21 of 28 (75%) of the antibodies. Interestingly, approximately the same, number (three to five) of immunodominant BoNT/A H_{c} peptide (nonconformational) epitopes are recognized by mouse and human polyclonal antibodies after immunization with pentavalent toxoid and by horse polyclonal antibodies after immunization with formaldehyde-inactivated BoNT/A (Atassi (1996) J. Protein Chem., 15: 691-699).

scFv binding epitopes 1 and 2 resulted in partial antagonism of toxin-induced neuroparalysis at the mouse neuromuscularjunction. When administered together, the two scFv had an additive effect, with the time to neuroparalysis increasing significantly. These results are consistent with the presence of two unique receptor binding sites on BoNT/A H_{c}. While the BoNT/A receptor(s) has not been formally identified, the results are consistent with those of ligand binding studies, which also indicate two classes of receptor binding sites on toxin, high and low affinity, and have led to a “dual receptor” model for toxin binding (Montecucco (1986) Trends Biochem. Sci. 11:314-317). Whether both of these sites are on H_{c}, however, is controversial. In two studies, BoNT/A H_{c} partially inhibited binding and neuromuscular paralysis (Black, et al. (1986) J. Cell Biol., 103:521-534; Black, et al. (1980) Am. J. Med., 69:567-570), whereas Daniels-Holgate et al. (1996) J. Neurosci. Res. 44:263-271, showed that BoNT/A H_{c} inhibited binding at motor nerve terminals but had no antagonistic effect on toxin-induced neuroparalysis at the mouse neuromuscular junction. Our results are consistent with the presence of two “productive” receptor binding sites on H_{c} which result in toxin internalization and toxicity. Differences in scFv potency may reflect differences in affinity of H_{c} for receptor binding sites or may reflect the greater than 10-fold difference in affinity of scFv for H_{c}. Finally, we have not formally shown that any of the scFv actually block binding of toxin to the cell surface. It is conceivable that the observed effect on time to neuroparalysis results from interference with a postbinding event.

scFv antagonism of toxin-induced neuroparalysis in the mouse hemidiaphragm assay was less than that (7.5-fold prolongation of time to neuroparalysis) observed for 2.0×10⁻⁹ M polyclonal equine antitoxin (PerImmune Inc.). This difference could be due to the necessity of blocking additional binding sites, differences in antibody affinity or avidity, or a cross-linking effect leading to aggregated toxin which cannot bind. Affinity of antibody binding is also likely to be an important factor, since the toxin binds with high affinity to its receptor (Williams et al. (1983) Eur. J. Biochem., 131: 437-445) and can be concentrated inside the cell by internalization. Of note, the most potent scFv has the highest affinity for H_{c}. Availability of other scFv described here, which recognize the same neutralizing epitope but with different K_{d}s, should help define the importance of affinity. These scFv, however, differ by many amino acids and may also differ in fine specificity, making interpretation of results difficult. Alternatively, mutagenesis combined with phage display can lead to the production of scFv which differ by only a few amino acids in sequence but vary by several orders of magnitude in affinity (Schier et al. (1996) J. Mol. Biol., 263: 551-567). The same approach can be used to increase antibody affinity into the picomolar range (Id.).

The “gold standard” for neutralization is protection of mice against the lethal effects of toxin coinjected with antibody. While the relationship between in vitro and in vivo results are consistent with the presence of two unique receptor binding sites on BoNT/A H_{c}. While the BoNT/A receptor(s) has not been formally identified, the results are consistent with those of ligand binding studies, which also indicate two classes of receptor binding sites on toxin, high and low affinity, and have led to a “dual receptor” model for toxin binding (Montecucco (1986) Trends Biochem. Sci. 11:314-317). Whether both of these sites are on H_{c}, however, is controversial. In two studies, BoNT/A H_{c} partially inhibited binding and neuromuscular paralysis (Black, et al. (1986) J. Cell Biol., 103:521-534; Black, et al. (1980) Am. J. Med., 69:567-570), whereas Daniels-Holgate et al. (1996) J. Neurosci. Res. 44:263-271, showed that BoNT/A H_{c} inhibited binding at motor nerve terminals but had no antagonistic effect on toxin-induced neuroparalysis at the mouse neuromuscular junction. Our results are consistent with the presence of two “productive” receptor binding sites on H_{c} which result in toxin internalization and toxicity. Differences in scFv potency may reflect differences in affinity of H_{c} for receptor binding sites or may reflect the greater than 10-fold difference in affinity of scFv for H_{c}. Finally, we have not formally shown that any of the scFv actually block binding of toxin to the cell surface. It is conceivable that the observed effect on time to neuroparalysis results from interference with a postbinding event.

scFv antagonism of toxin-induced neuroparalysis in the mouse hemidiaphragm assay was less than that (7.5-fold prolongation of time to neuroparalysis) observed for 2.0×10⁻⁹ M polyclonal equine antitoxin (PerImmune Inc.). This difference could be due to the necessity of blocking additional binding sites, differences in antibody affinity or avidity, or a cross-linking effect leading to aggregated toxin which cannot bind. Affinity of antibody binding is also likely to be an important factor, since the toxin binds with high affinity to its receptor (Williams et al. (1983) Eur. J. Biochem., 131: 437-445) and can be concentrated inside the cell by internalization. Of note, the most potent scFv has the highest affinity for H_{c}. Availability of other scFv described here, which recognize the same neutralizing epitope but with different K_{d}s, should help define the importance of affinity. These scFv, however, differ by many amino acids and may also differ in fine specificity, making interpretation of results difficult. Alternatively, mutagenesis combined with phage display can lead to the production of scFv which differ by only a few amino acids in sequence but vary by several orders of magnitude in affinity (Schier et al.(1996) J. Mol. Biol., 263: 551-567). The same approach can be used to increase antibody affinity into the picomolar range (Id.).

The “gold standard” for neutralization is protection of mice against the lethal effects of toxin coinjected with antibody. While the relationship between in vitro and in vivo protection has not been formally established, equine antitoxin potentially neutralizes toxin in both types of assays (see above and Hatheway et al. (1 984) J. Infect. Dis., 150: 407-412). It is believed that this relationship holds for the scFv reported here, and this can be verified experimentally.

Such studies are not possible with small (25-kDa) scFv antibody fragments. The small size of scFv leads to rapid redistribution (the half-life at a phase is 2.4 to 12 min) and clearance (the half-life at β phase is 1.5 to 4 h) and antibody levels which rapidly become undetectable (Huston, et al.,(1996) J. Nucl. Med. 40: 320; Schier et al. (1995) Immunotechnology, 1: 73-81), while toxin levels presumably remain high (Hildebrand, et al. (1961) Proc. Soc. Exp. Biol. Med. 107-284-289). Performance of in vivo studies will be facilitated by the construction of complete IgG molecules from the V_{H} and V_{L} genes of scFv. Use of human constant regions will yield chimeric antibodies less immunogenic than murine monoclonals and much less immunogenic than currently used equine antitoxin. Immunogenicity can be further reduced by CDR grafting to yield humanized antibodies.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An isolated antibody that specifically binds to an epitope specifically bound by an antibody expressed by a clone selected from the group consisting of clone S25, clone C25, clone C39, clone 1C6, and clone 1F3, wherein said antibody binds to and neutralizes botulinum neurotoxin type A (BoNT/A).

2. The antibody of claim 1, wherein said clone is S25.

3. The antibody of claim 1, wherein said clone is C25 or C39.

4. The antibody of claim 1, wherein said clone is 1C6.

5. The antibody of claim 1, wherein said clone is 1F3.

6. The antibody of claim 1, wherein said antibody comprises at least two variable heavy (V_{H}) complementarity determining regions (CDRs) listed in Table 4.

7. The antibody of claim 6, wherein said antibody comprises at three variable heavy (V_{H}) complementarity determining regions (CDRs) listed in Table 4.

8. The antibody of claim 1, wherein said antibody further comprises a variable light (V_{L}) complementarity determining region (CDR) listed in Table 4.

9. The antibody of claim 8, wherein said antibody comprises at least two variable light (V_{L}) complementarity determining regions (CDRs) listed in Table 4.

10. The antibody of claim 9, wherein said antibody comprises three variable light (V_{L}) complementarity determining regions (CDRs) listed in Table 4.

11. The antibody of claim 1, wherein said antibody is an antibody expressed by a clone selected from the group consisting of a clone listed in Table 4.

12. The antibody of claim 1, wherein said antibody is a single chain Fv (scFv).

13. The antibody of claim 1, wherein said antibody is a Fab.

14. The antibody of claim 1, wherein said antibody is a (Fab′)₂.

15. The antibody of claim 1, wherein said antibody is a (scFv′)₂.

16. The antibody of claim 15, wherein said antibody is a fusion protein of two scFv fragments.

17. The antibody of claim 1, wherein said antibody comprises a framework region listed in Table 4.

18. The antibody of claim 17, wherein said framework is a variable heavy (V_{H}) frame work region listed in Table 4.

19. The antibody of claim 17, wherein said framework is a variable light (V_{L}) frame work region listed in Table 4.

20. The antibody of claim 18, wherein said antibody comprises at least two variable heavy (V_{H}) framework regions listed in Table 4.

21. The antibody of claim 19, wherein said antibody comprises at least two variable light (V_{L}) framework regions listed in Table 4.

22. The antibody of claim 18, wherein said antibody comprises a variable heavy (V_{H}) region listed in Table 4.

23. The antibody of claim 19, wherein said antibody comprises a variable light (V_{L}) region listed in Table 4.

24. An isolated anti-botulinum neurotoxin type A (anti-BoNT/A) antibody, said antibody comprising a variable heavy (V_{H}) complementarity determining region (CDR) listed in Table 4 and wherein said antibody specifically binds to and neutralizes a botulinum neurotoxin type A.

25. The antibody of claim 24, wherein said antibody comprises at least two variable heavy (V_{H}) complementarity determining regions (CDRs) listed in Table 4.

26. The antibody of claim 25, wherein said antibody comprises at three variable heavy (V_{H}) complementarity determining regions (CDRs) listed in Table 4.

27. The antibody of claim 24, wherein said antibody further comprises a variable light (V_{L}) complementarity determining region (CDR) listed in Table 4.

28. The antibody of claim 27, wherein said antibody comprises at least two variable light (V_{L}) complementarity determining regions (CDRs) listed in Table 4.

29. The antibody of claim 28, wherein said antibody comprises three variable light (V_{L}) complementarity determining regions (CDRs) listed in Table 4.

30. The antibody of claim 24, wherein said antibody is an antibody expressed by a clone selected from the group consisting of a clone listed in Table 4.

31. The antibody of claim 24, wherein said antibody is a single chain Fv (scFv).

32. The antibody of claim 24, wherein said antibody is a Fab.

33. The antibody of claim 24, wherein said antibody is a (Fab′)₂.

34. The antibody of claim 24,wherein said antibody is a (scFv′)₂.

35. The antibody of claim 34, wherein said antibody is a fusion protein of two scFv fragments.

36. The antibody of claim 24, wherein said antibody comprises a framework region listed in Table 4.

37. The antibody of claim 36, wherein said framework is a variable heavy (V_{H}) frame work region listed in Table 4.

38. The antibody of claim 36, wherein said framework is a variable light (V_{L}) frame work region listed in Table 4.

39. The antibody of claim 37, wherein said antibody comprises at least two variable heavy (V_{H}) framework regions listed in Table 4.

40. The antibody of claim 38, wherein said antibody comprises at least two variable light (V_{L}) framework regions listed in Table 4.

41. The antibody of claim 37, wherein said antibody comprises a variable heavy (V_{H}) region listed in Table 4.

42. The antibody of claim 38, wherein said antibody comprises a variable light (V_{L}) region listed in Table 4.

43. The antibody of claim 24, wherein antibody specifically binds to an epitope specifically bound by an antibody expressed by a clone selected from the group consisting of clone S25, clone C25, clone C39, clone 1C6, and clone 1F3.

44. A method of neutralizing a botulinum neurotoxin type A (BoNT/A), said method comprising contacting said botulinum neurotoxin type A with a first anti-botulinum neurotoxin type A (anti-BoNT/A) antibody, said antibody comprising a variable heavy (V_{H}) complementarity determining region (CDR) listed in Table 4 said antibody having a specificity and affinity such that it specifically binds to binds to and neutralizes said botulinum neurotoxin type A.

45. The method of claim 44, further comprising contacting said botulinum neurotoxin type A with a second anti-botulinum neurotoxin type A (anti-BoNT/A) antibody, said antibody comprising a variable heavy (V_{H}) complementarity determining region (CDR) listed in Table 4 said antibody having a specificity and affinity such that it specifically binds to binds to and neutralizes said botulinum neurotoxin type A, wherein said second anti-botulinum neurotoxin type A (anti-BoNT/A) antibody binds to a different epitope than said first anti-botulinum neurotoxin type A (anti-BoNT/A) antibody.

46. The method of claim 44, wherein said antibody comprises at least two variable heavy (V_{H}) complementarity determining regions (CDRs) listed in Table 4.

47. The method of claim 46, wherein said antibody comprises at three variable heavy (V_{H}) complementarity determining regions (CDRs) listed in Table 4.

48. The method of claim 44, wherein said antibody further comprises a variable light (V_{L}) complementarity determining region (CDR) listed in Table 4.

49. The method of claim 48, wherein said antibody comprises at least two variable light (V_{L}) complementarity determining regions (CDRs) listed in Table 4.

50. The method of claim 49, wherein said antibody comprises three variable light (V_{L}) complementarity determining regions (CDRs) listed in Table 4.

51. The method of claim 44, wherein said antibody is an antibody expressed by a clone listed in Table 4.

52. The method of claim 44, wherein said antibody is a single chain Fv (scFv).

53. The method of claim 44, wherein said antibody is a Fab.

54. The method of claim 44, wherein said antibody is a (Fab′)₂.

55. The method of claim 44, wherein said antibody is a (scFv′)₂.

56. The method of claim 55, wherein said antibody is a fusion protein of two scFv fragments.

57. The method of claim 44, wherein said antibody comprises a framework region listed in Table 4.

58. The method of claim 57, wherein said framework is a variable heavy (V_{H}) frame work region listed in Table 4.

59. The method of claim 57, wherein said framework is a variable light (V_{L}) frame work region listed in Table 4.

60. The method of claim 58, wherein said antibody comprises at least two variable heavy (V_{H}) framework regions listed in Table 4.

61. The method of claim 59, wherein said antibody comprises at least two variable light (V_{L}) framework regions listed in Table 4.

62. The method of claim 58, wherein said antibody comprises a variable heavy (V_{H}) region listed in Table 4.

63. The method of claim 59, wherein said antibody comprises a variable light (V_{L}) region listed in Table 4.

64. A polypeptide comprising botulinum neurotoxin type A (BoNT/A) neutralizing epitope, said neutralizing epitope comprising an epitope specifically bound by an antibody expressed by a clone selected from the group consisting of clone S25, clone C25, clone C39, clone 1C6, and clone 1F3, wherein said polypeptide is not a full-length botulinum neurotoxin H_{c} fragment.

65. The polypeptide of claim 64, wherein said polypeptide is a fragment of BoNT/A H_{C} having a length of at least 8 amino acids.

66. The polypeptide of claim 64, wherein said clone is S25.

67. The polypeptide of claim 64, wherein said clone is C25 or C39.

68. The polypeptide of claim 64, wherein said clone is 1C6.

69. The polypeptide of claim 64, wherein said clone is 1F3.

70. A method of making a botulinum neurotoxin type A antibody (anti-BoNT/A) that neutralizes BoNT/A, said method comprising:
contacting a plurality of antibodies with a an epitope specifically bound by an antibody expressed by a clone selected from the group consisting of clone S25, clone C25, clone C39, clone 1C6, and clone 1F3; and isolating an antibody that specifically binds to said epitope.

71. The method of claim 70, wherein said clone is S25.

72. The method of claim 70, wherein said clone is C25 or C39.

73. The method of claim 70, wherein said clone is 1C6.

74. The method of claim 70, wherein said clone is 1F3.

75. The method of claim 70, wherein said plurality of antibodies are antibodies displayed on a surface protein of a phage.

76. The method of claim 70, wherein said plurality of antibodies are antibodies in serum from a mammal.

77. The method of claim 70, wherein said plurality of antibodies are antibodies expressed by hybridomas.
